(19) United States
(12) Patent Application Publication
(10) Pub. No.: US 2005/0096350 A1
(43) Pub. Date: May 5, 2005

(54) Halo substituted benzo[b]thiophenes as therapeutic agents

(76) Inventors: Michael K. Connolly, Ypsilanti, MI (US); Rocco Dean Gogliotti, Pinckney, MI (US); Clarence R. Hurt, San Ramon, CA (US); Gregory Adam Reichard, Ann Arbor, MI (US); Melean Visnick, Ann Arbor, MI (US)

Correspondence Address: WARNER-LAMBERT COMPANY, 2800 PLYMOUTH RD, ANN ARBOR, MI, 48105, US

(21) Appl. No.: 10/935,356

(22) Filed: Sep. 7, 2004

Related U.S. Application Data
(60) Provisional application No. 60/500,038, filed on Sep. 4, 2003.

(51) Int. Cl. C07D 49/14; A61K 31/454; A61K 31/41

(52) U.S. Cl. 514/320; 514/381; 546/202; 548/253

(57) ABSTRACT

The present invention provides benzo[b]thiophenes of Formula I: 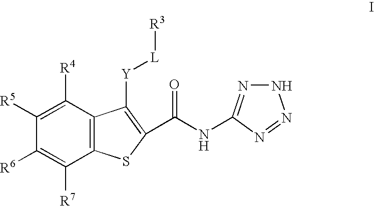
wherein R³, R⁴, R⁵, R⁶, R⁷, Y, and L have any of the values defined therefor in the specification, and pharmaceutically acceptable salts thereof, that are useful as agents in the treatment of diseases and conditions, including inflammatory diseases, cardiovascular diseases, and cancers. Also provided are pharmaceutical compositions comprising one or more compounds of Formula I.

### CROSS-REFERENCES TO RELATED APPLICATIONS

This application claims the benefit of U.S. Provisional Patent application Ser. No. 60/500,038, filed on Sep. 4, 2003, the teachings of which are herein incorporated by reference.

### BACKGROUND OF THE INVENTION

Phosphoinositide-3-kinases (PI3Ks) are a family of lipid kinases that phosphorylate phosphoinositols on the 3′-OH to generate PI-3-P (phosphatidylinositol 3-phosphate), PI-3,4-P2 and PI-3,4,5-P3. One class of PI3Ks are stimulated by growth factors. A separate class of PI3Ks are activated by G-protein coupled receptors and include PI3Kγ. The growth-factor stimulated PI3Ks (e.g., PI3Kα) have been implicated in cellular proliferation and cancer. PI3Kγ has been demonstrated to be involved in signaling cascades. For example, PI3Kγ is activated in response to ligands such as C5a, fMLP, ADP, and IL-8. In addition, PI3Kγ has been implicated in immune diseases (Hirsch et al. Science 2000;287:1049-1053). PI3Kγ null macrophages show a reduced chemotactic response and a reduced ability to fight inflammation (Hirsch et al., 2000, supra). Furthermore, PI3Kγ has also been implicated in thrombolytic diseases (e.g., thromboembolism, ischemic diseases, heart attacks, and stroke) (Hirsch et al. FASEB J. 2000; 15(11):2019-2021; and Hirsch et al. FASEB J., Jul. 9 2001;10.1096/fj.00-0810fje (cited herein as Hirsch et al., 2001).

Inhibitors of members of the PI3Ks are being developed for the treatment of human disease (see e.g., WO 01/81346; WO 01/53266; and WO 01/83456). There is a need for additional compounds that can inhibit PI3Ks for use as pharmaceutical agents.

### SUMMARY OF THE INVENTION

In one aspect, the present invention provides for benzo[b]thiophenes of formula I: 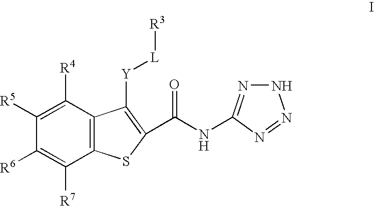
or a pharmaceutically acceptable salt thereof; wherein
Y is O or S;
two of R⁴, R⁵, R⁶, and R⁷ are hydrogen:
one of R⁴, R⁵, R⁶, and R⁷ is selected from the group consisting of: methoxy, C₁-C₃-alkyl-O, CH₂FO, CHF₂O, CF₃O, CF₃CH₂O, or cyclopropyloxy;
one of R⁴, R⁵, R⁶, and R⁷is F, I, Br, or Cl;
L is absent, a C₁-C₄ alkylene, or 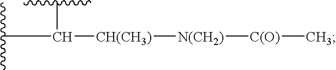
R³ is:
   (a) selected from the group consisting of: a C₃-C₈ cycloalkyl, a 5 or 6-membered heterocycloalkyl, a tetrahydropyranyl, and a piperidinyl;
      the C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃; or
   (b) a phenyl group;
      the phenyl group may be optionally substituted with: 1 to 3 substituents independently selected from the group consisting of:
         Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and —(O)ₘ—C₃-C₈ cycloalkyl, n is 0, 1 or 2; and m is 0 or 1.

In certain embodiments of Formula I, L is absent or is a C₁₋₄alkylene; two of R⁴, R⁶, and R⁷ are hydrogen; R⁵ is selected from the group consisting of: methoxy, C₁-C₃-alkyl-O, CH₂FO, CHF₂O, CF₃O, CF₃CH₂O, or cyclopropyloxy; and one of R⁴, R₆, and R⁷ is F, Br, or Cl—a compound of Formula Ia.

In certain embodiments of Formula II, R⁵ is methoxy; R⁶ is F; Y is O; and R³ is a phenyl group; the phenyl group may be optionally substituted with:
1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and a —(O)ₘ—C₃-C₈ cycloalkyl; n is 0, 1 or 2; and m is 0 or 1—a compound of Formula IIa:

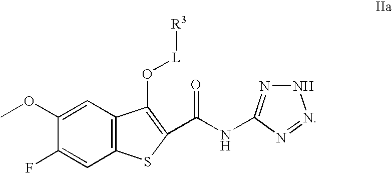

Examples of a compound of Formula IIa include, but are not limited to:
6-Fluoro-3-(4-isopropyl-phenoxy)-5-methoxy-benzo[b]thiophene-2-carboxylic acid (2H-tetrazol-5-yl)-amide;
6-Fluoro-5-methoxy-3-phenoxy-benzo[b]thiophene-2-carboxylic acid iminomethyl-amide;
3-(4-Cyclohexyl-phenoxy)-6-fluoro-5-methoxy-benzo[b]thiophene-2-carboxylic acid (2H-tetrazol-5-yl)-amide;
3-(3-Ethyl-phenoxy)-6-fluoro-5-methoxy-benzo[b]thiophene-2-carboxylic acid (2H-tetrazol-5-yl)-amide;
3-(4-Chloro-phenoxy)-6-fluoro-5-methoxy-benzo[b]thiophene-2-carboxylic acid (2H-tetrazol-5-yl)-amide; and
3-(4-Cyclopentyloxy-phenoxy)-6-fluoro-5-methoxy-benzo[b]thiophene-2-carboxylic acid (2H-tetrazol-5-yl)-amide.

In certain embodiments of Formula II, R⁵ is methoxy; R⁶ is F; Y is O; and R³ is a group selected from the group consisting of a C₃-C₈ cycloalkyl, a 5 or 6-membered heterocycloalkyl, a tetrahydropyranyl, and a piperidinyl;
the C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃—a compound of Formula IIb:

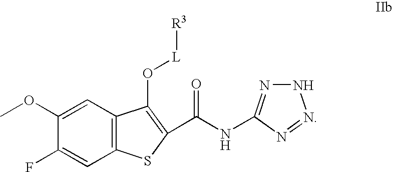

Examples of a compound of Formula IIb include, but are not limited to:
3-(3,5-Dimethyl-cyclohexyloxy)-6-fluoro-5-methoxy-benzo[b]thiophene-2-carboxylic acid (2H-tetrazol-S-yl)-amide;
3-Cycloheptyloxy-6-fluoro-5-methoxy-benzo[b]thiophene-2-carboxylic acid (2H-tetrazol-5-yl)-amide;
cis-(±)-6-Fluoro-5-methoxy-3-(3-methyl-cyclohexyloxy)-benzo[b]thiophene-2-carboxylic acid (2H-tetrazol-5-yl)-amide;
6-Fluoro-5-methoxy-3-(3,3,5-trimethyl-cyclohexyloxy)-benzo[b]thiophene-2-carboxylic acid (2H-tetrazol-5-yl)-amide;
3-(3,3-Dimethyl-cyclohexyloxy)-6-fluoro-5-methoxy-benzo[b]thiophene-2-carboxylic acid (2H-tetrazol-5-yl)-amide;
3-Cyclohexyloxy-6-fluoro-5-methoxy-benzo[b]thiophene-2-carboxylic acid (2H-tetrazol-5-yl)-amide;
6-Fluoro-5-methoxy-3-(1-methyl-cyclopropylmethoxy)-benzo[b]thiophene-2-carboxylic acid (2H-tetrazol-5-yl)-amide; and
4-[6-Fluoro-5-methoxy-2-(2H-tetrazol-5-ylcarbamoyl)-benzo[b]thiophen-3-yloxy]-piperidine-1-carboxylic acid tert-butyl ester.

In certain embodiments of Formula II, R⁵ is methoxy; R⁶is F; Y is S; and R³ is a phenyl group; the phenyl group may be optionally substituted with: 1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and a —(O)ₘ—C₃-C₈ cycloalkyl, n is 0, 1 or 2; and m is 0 or 1—a compound of Formula IIc:

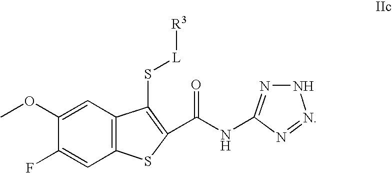

An example of a compound of Formula IIc is 6-Fluoro-5-methoxy-3-phenylsulfanyl-benzo[b]thiophene-2-carboxylic acid (1H-tetrazol-5-yl)-amide.

In certain embodiments of Formula II, R⁵ is methoxy; R⁶ is F; Y is S; and R³ is a group selected from the group consisting of a C₃-C₈ cycloalkyl, a 5 or 6-membered heterocycloalkyl, a tetrahydropyranyl, and a piperidinyl;
the C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃—a compound of Formula IId.

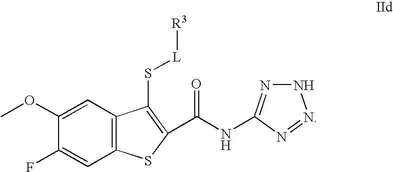

Examples of a compound of Formula IId include, but are not limited to:
3-Cyclopentylsulfanyl-6-fluoro-5-methoxy-benzo[b]thiophene-2-carboxylic acid (1H-tetrazol-5-yl)-amide; and
3-Cyclohexylsulfanyl-6-fluoro-5-methyl-benzo[b]thiophene-2-carboxylic acid (1H-tetrazol-5-yl)-amide.

In certain embodiments of Formula I, R⁶ and R⁷ are H; and R⁴ is F, Br, or Cl—a compound of Formula III:

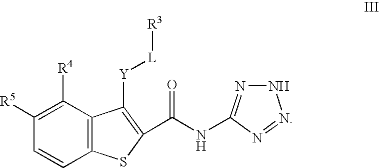

Examples of a compound of Formula IId include, but are not limited to:
3-Cyclohexylsulfanyl-4-fluoro-5-methoxy-benzo[b]thiophene-2-carboxylic acid (1H-tetrazol-5-yl)-amide;
4-Fluoro-3-(4-hydroxy-phenylsulfanyl)-5-methoxy-benzo[b]thiophene-2-carboxylic acid (1H-tetrazol-5-yl)-amide; and
4-Fluoro-3-(3-hydroxy-phenylsulfanyl)-5-methoxy-benzo[b]thiophene-2-carboxylic acid (1H-tetrazol-5-yl)-amide.

In certain embodiments of Formula III, R⁵ is methoxy; R⁴ is F; and R³ is a group selected from the group consisting of a C₃-C₈ cycloalkyl, a 5 or 6-membered heterocycloalkyl, a tetrahydropyranyl, and a piperidinyl;
the C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃—a compound of Formula IIIa:

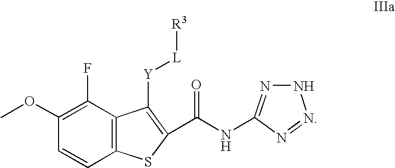

In certain embodiments of Formula I, L is absent or is a C₁₋₄alkylene, two of R⁴, R⁵, and R⁷ are hydrogen; R⁶ is selected from the group consisting of:
methoxy, C₁-C₃-alkyl-O, CH₂FO, CHF₂O, CF₃O, CF₃CH₂O, or cyclopropyloxy; one of R⁴, R⁶, and R⁷ is F, Br, or Cl—a compound of Formula IV.

In certain embodiments of Formula IV, R⁴ and R⁵ are H; and R⁷ is F, Br, or Cl—a compound of Formula IVa.

In certain embodiments of Formula IV, R⁶ is methoxy; R⁷ is F; and R³ is a phenyl group;
the phenyl group may be optionally substituted with:
1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and a —(O)ₘ—C₃-C₈ cycloalkyl, n is 0, 1 or 2; and m is 0 or 1—a compound of Formula IVb:

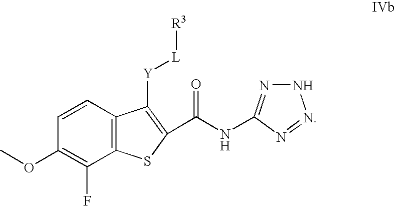

In certain embodiments of Formula IV, R⁶ is methoxy; R⁷ is F; Y is S; and R³ is a group selected from the group consisting of a C₃-C₈ cycloalkyl, a 5 or 6-membered heterocycloalkyl, a tetrahydropyranyl, and a piperidinyl;
the C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃—a compound of Formula IVc:

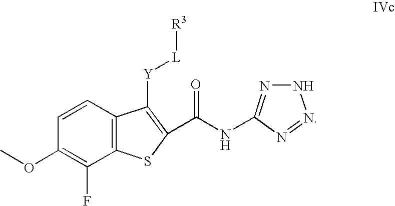

Examples of a compound of Formula IVc include, but are not limited to:
3-Cyclopentylsulfanyl-7-fluoro-6-methoxy-benzo[b]thiophene-2-carboxylic acid (1H-tetrazol-5-yl)-amide; and
3-Cyclohexylsulfanyl-7-fluoro-6-methoxy-benzo[b]thiophene-2-carboxylic acid (1H-tetrazol-5-yl)-amide.

In another aspect, the invention provides for pharmaceutical compositions that comprise a therapeutically effective amount of a compound of Formulas I-IVc and a pharmaceutically acceptable carrier. In certain embodiments, these compositions are useful in the treatment of a PI3K-mediated disorder or condition. The compounds of the invention can also be combined in a pharmaceutical composition that also comprise other compounds that are useful for the treatment of cancer, a thrombolytic disease, heart disease, stroke, an inflammatory disease such as rheumatoid arthritis, or another PI3K-mediated disorder.

In another aspect, the present invention provides for methods of treating a subject suffering from a PI3K-mediated disorder or condition comprising: administering, to a subject suffering from a PI3K-mediated condition or disorder, a pharmaceutical composition comprising a therapeutically effective amount of a compound of Formulas I-IVc and a pharmaceutically acceptable carrier. In certain embodiments, the PI3K-mediated condition or disorder is selected from the group consisting of: rheumatoid arthritis, osteoarthritis, psoriatic arthritis, ankylosing spondylitis, psoriasis, inflammatory diseases, and autoimmune diseases. In other embodiments, the PI3K-mediated condition or disorder is selected from the group consisting of: cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease. In still other embodiments, the PI3K-mediated condition or disorder is selected from the group consisting of: cancer, colon cancer, glioblastoma, endometrial carcinoma, hepatocellular cancer, lung cancer, melanoma, renal cell carcinoma, thyroid carcinoma, cell lymphoma, lymphoproliferative disorders, small cell lung cancer, squamous cell lung carcinoma, glioma, breast cancer, prostate cancer, ovarian cancer, cervical cancer, and leukemia. In yet another embodiment, the PI3K-mediated condition or disorder is selected from the group consisting of: type II diabetes. In still other embodiments, the PI3K-mediated condition or disorder is selected from the group consisting of: respiratory diseases, bronchitis, asthma, and chronic obstructive pulmonary disease. In certain embodiments, the subject is a human.

### DEFINITIONS

As used herein, the following terms have the meanings ascribed to them unless specified otherwise.

A “PI3K-mediated disorder or condition” is characterized by the participation of one or more PI3Ks or a PI3P phosphatase, (e.g., PTEN, etc.) in the inception, manifestation of one or more symptoms or disease markers, severity, or progression of a disorder or condition. PI3K-mediated disorders and conditions include, but are not limited to: rheumatoid arthritis, osteoarthritis, psoriatic arthritis, psoriasis, inflammatory diseases, pulmonary fibrosis, autoimmune diseases, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, coronary artery disease, cancer, breast cancer, gliobastoma, endometrial carcinoma, hepatocellular carcinoma, colon cancer, lung cancer, melanoma, renal cell carcinoma, thyroid carcinoma, small cell lung cancer, squamous cell lung carcinoma, glioma, prostate cancer, ovarian cancer, cervical cancer, leukemia, cell lymphoma, lymphoproliferative disorders, type II diabetes, respiratory diseases, bronchitis, asthma, and chronic obstructive pulmonary disease.

A PI3K is an enzyme that is able to phosphorylate the 3′-OH of a phosphoinositol to generate PI3P. PI3Ks include, but are not limited to, PI3Kα, PI3Kβ, PI3Kγ, and PI3Kδ. A PI3K typically comprises at least one catalytic subunit (e.g., p110γ), and may further comprise a regulatory subunit (e.g., p101, etc.).

The term “alkyl group” or “alkyl” includes straight and branched carbon chain radicals. The term “alkylene” refers to a diradical of an unsubstituted or substituted alkane. For example, a “C₁₋₆ alkyl” is an alkyl group having from 1 to 6 carbon atoms. Examples of straight-chain alkyl groups include, but are not limited to, methyl, ethyl, n-propyl, n-butyl, n-pentyl, n-hexyl, n-heptyl, n-octyl, n-nonyl, n-decyl, etc. Examples of branched-chain alkyl groups include, but are not limited to, isopropyl, tert-butyl, isobutyl, etc. Examples of alkylene groups include, but are not limited to, —CH₂—, —CH₂—CH₂—, —CH₂—CH(CH₃)—CH₂—, and —(CH₂)₁₋₆. Alkylene groups can be substituted with groups as set forth below for alkyl.

Moreover, the term alkyl includes both “unsubstituted alkyls” and “substituted alkyls,” the latter of which refers to alkyl moieties having substituents replacing a hydrogen on one or more carbons (e.g., replacing a hydrogen on 1, 2, 3, 4, 5, or 6 carbons) of the hydrocarbon backbone. Such substituents can include, but are not limited to, C₂-C₆-alkenyl, C₂-C₆-alkynyl, halo, I, Br, Cl, F, —OH, —COOH, sulfhydryl, (C₁-C₆-alkyl)S—, C₁-C₆-alkylsulfinyl, nitro, cyano, trifluoromethyl, —NH₂, ═O, ═S, ═N—CN, ═N—OH, —OCH₂F, —OCHF₂, —OCF₃, —SCF₃, —SO₂—NH₂, C₁-C₆-alkoxy, —C(O)O—(C₁-C₆ alkyl), —O—C(O)—(C₁-C₆ alkyl), —C(O)—NH₂, —C(O)—N(H)—C₁-C₆ alkyl, —C(O)—N(C₁-C₆ alkyl)₂, —OC(O)—NH₂, —C(O)—H, —C(O)—(C₁-C₆ alkyl), —C(S)—(C₁-C₆ alkyl), —NR⁷⁰R⁷², where R⁷⁰ and R⁷² are each independently selected from H, C₁-C₆-alkyl, C₂-C₆-alkenyl, C₂-C₆-alkynyl, and C(O)—C₁-C₆-alkyl.

Typical substituted alkyl groups thus are aminomethyl, 2-nitroethyl, 4-cyanobutyl, 2,3-dichloropentyl, and 3-hydroxy-5-carboxyhexyl, 2-aminoethyl, pentachloroethyl, trifluoromethyl, 2-diethylaminoethyl, 2-dimethylaminopropyl, ethoxycarbonylmethyl, methanylsulfanylmethyl, methoxymethyl, 3-hydroxypentyl, 2-carboxybutyl, 4-chlorobutyl, and pentafluoroethyl.

“Halo” includes fluoro, chloro, bromo, and iodo.

“Alkenyl” means straight and branched hydrocarbon radicals having 2 or more carbon atoms and comprising at least one carbon-carbon double bond and includes ethenyl, 3-buten-1-yl, 2-ethenylbutyl, 3-hexen-1-yl, and the like. The term “alkenyl” is intended to include both substituted and unsubstituted alkenyl groups. A “C₂-C₆-alkenyl” is an alkenyl group having from from 2 to 6 carbon atoms. Alkenyl groups can be substituted with groups such as those set out above for alkyl.

“Alkynyl” means straight and branched hydrocarbon radicals having 2 or more carbon atoms and comprising at least one carbon-carbon triple bond and includes ethynyl, 3-butyn-1-yl, propynyl, 2-butyn-1-yl, 3-pentyn-1-yl, and the like. The term “alkynyl” is intended to include both substituted and unsubstituted alkynyl groups. Alkynyl groups can be substituted with groups such as those set out above for alkyl. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

The term “C₃-C₈cycloalkyl” refers to a cycloalkyl group containing from 3 to 8 carbons. Thus, the term “C₃-C₈cycloalkyl” encompasses a monocyclic cycloalkyl group containing from 3 to 8 carbons and a bicyclic cycloalkyl group containing from 6 to 8 carbons. Examples of “C₃-C₈cycloalkyls” includes, but are not limited to, cyclopropyl, cyclobutyl, cyclopentyl, cyclohexyl, cycloheptyl, cyclooctyl, and bicyclo[2.2.1]heptanyl; the cycloalkyl group may optionally contain 1 or 2 double bonds (i.e., a cycloalkylenyl) including, but not limited to, cyclopentenyl, cyclohexenyl, and cycloheptenyl. Cycloalkyl groups groups can be substituted with groups such as those set out above for alkyl. In certain embodiments, a “C₃-C₈cycloalkyl” may be optionally substituted with 1, 2, 3, or 4 methyls, or —C(O)—O—C(CH₃)₃. Examples of substituted cycloalkyl groups include, but are not limited to, 2-methyl-cyclohexyl, 3-methyl-cyclohexyl, 3,5-dimethyl-cyclohexyl, 2,3,5-Trimethyl-cyclohexyl, and 4-methyl-cyclohexyl.

The phrase “5 or 6-membered heterocycloalkyl” means a stable cyclic group having carbon atoms and 1 to 3 heteroatoms independently selected from S, N or O, wherein when two O atoms or one O atom and one S atom are present, the two O atoms or one O atom and one S atom are not bonded directly to each other, respectively. Optionally, a 5 or 6-membered heterocycloalkyl may contain 1 or 2 carbon-carbon or carbon-nitrogen double bonds. Illustrative examples of 5 or 6-membered heterocycloalkyl include tetrahydrofuran-3-yl, morpholin-4-yl, 2-thiacyclohex-1-yl, 2-oxo-2-thiacyclohex-1-yl, 2,2-dioxo-2-thiacyclohex-1-yl, piperidinyl, tetrahydropyranyl, and 4-methyl-piperazin-2-yl.

Illustrative examples of substituted 5 or 6-membered heterocycloalkyls include 2,2-dimethyl-tetrahydrofuran-3-yl and

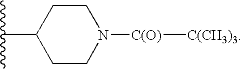

Unless otherwise indicated, the foregoing heterocycloalkyls can be C-attached or N-attached where such is possible and which results in the creation of a stable structure. For example, piperidinyl can be piperidin-1-yl (N-attached) or piperidin-4-yl (C-attached).

Embraced within the term “5 or 6 membered heterocycloalkyl” are 5 membered rings having one carbon-carbon or one carbon-nitrogen double bond in the ring (e.g., 2-pyrrolinyl, 3-pyrrolinyl, etc.) and 6 membered rings having one carbon-carbon or one carbon-nitrogen double bond in the ring (e.g., dihydro-2H-pyranyl, 1,2,3,4-tetrahydropyridine, 3,4-dihydro-2H-[1,4]oxazine, etc.).

A “5-membered heterocycloalkyl” is a stable 5-membered, monocyclic cycloalkyl ring having from 2 to 4 carbon atoms and from 1 to 3 heteroatoms selected from the group consisting of: 1 O; 1 S; 1 N; 2 N; 3 N; 1 S and 1 N; 1 S, and 2 N; 1 O and 1 N; and 1 O and 2 N. Illustrative examples of stable 5-membered heterocycloalkyls include tetrahydrofuranyl, dihydrofuranyl, tetrahydrothienyl, dihydrothienyl, imidazolidinyl, oxazolidinyl, imidazolinyl, isoxazolidinyl, pyrrolidinyl, 2-pyrrolinyl, and 3-pyrrolinyl.

A “6-membered heterocycloalkyl” is a stable 6-membered, monocyclic cycloalkyl ring having from 3 to 5 carbon atoms and from 1 to 3 heteroatoms selected from the group consisting of: 1 O; 2 O;1 S; 2 S; 1 N; 2 N; 3 N; 1 S, 1 O, and 1 N; 1 S and 1 N; 1 S and 2 N; 1 S and 1 O; 1 S and 2 O; 1 O and 1 N; and 1 O and 2 N. Illustrative examples of stable 6-membered heterocycloalkyls include tetrahydropyranyl, dihydropyranyl, dioxanyl, 1,3-dioxolanyl, 1,4-dithianyl, hexahydropyrimidine, morpholinyl, piperazinyl, piperidinyl, 2H-pyranyl, 4H-pyranyl, pyrazolidinyl, pyrazolinyl, 1,2,3,6-tetrahydropyridinyl, tetrahydrothiopyranyl, 1,1-dioxo-hexahydro-1λ⁶-thiopyranyl, 1,1-dioxo-1λ⁶-thiomorpholinyl, thiomorpholinyl, thioxanyl, and trithianyl.

The term “5 or 6-membered heterocycloalkyl” includes saturated and unsaturated “5 or 6-membered heterocycloalkyls.” “5 or 6-membered heterocycloalkyls” may be substituted with 1 to 4 groups such as those set out above for alkyl. In certain embodiments, a 5 or 6 membered heterocycloalkyl may be optionally substituted with 1, 2, 3, or 4 methyls, or —C(O)—O—C(CH₃)₃, where possible.

The term “phenyl” refers to the monoradical radical C₆H₅—, from benzene. A phenyl group may be optionally substituted at one to four positions with a substituent such as, but not limited to, those substituents described above for alkyl. In certain embodiments, a phenyl group may be optionally substituted with 1 to 3 substituents independently selected from the group consisting of: Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and —(O)ₘ—C₃-C₈ cycloalkyl; or n is 0, 1, or 2; and m is 0 or 1.

Typical substituted phenyl groups include, but are not limited to, 3-chlorophenyl, 2,6-dibromophenyl, 2,4,6-tribromophenyl, 2,6-dichlorophenyl, 4-trifluoromethylphenyl, 3,5-dihydroxyphenyl, 3-methyl-phenyl, 4-methyl-phenyl, 3,5-dimethyl-phenyl, 3,4,5-trimethoxy-phenyl, 3,5-dimethoxy-phenyl, 3,4-dimethoxy-phenyl, 3-methoxy-phenyl, 4-methoxy-phenyl, 4-tert-butyl-phenyl, 3,5-difluoro-phenyl, 4-chloro-phenyl, 3-trifluoromethyl-phenyl, 3,5-dichloro-phenyl, 2-methoxy-5-methyl-phenyl, 2-fluoro-5-methyl-phenyl, 4-chloro-2-trifluoromethyl-phenyl, and the like.

Some of the compounds in the present invention may exist as stereoisomers, including enantiomers, diastereomers, and geometric isomers. Geometric isomers include compounds of the present invention that have alkenyl groups, which may exist as entgegen or zusammen conformations, in which case all geometric forms thereof, both entgegen and zusammen, cis and trans, and mixtures thereof, are within the scope of the present invention. Some compounds of the present invention have cycloalkyl groups, which may be substituted at more than one carbon atom, in which case all geometric forms thereof, both cis and trans, and mixtures thereof, are within the scope of the present invention. All of these forms, including (R), (S), epimers, diastereomers, cis, trans, syn, anti, (E), (Z), tautomers, and mixtures thereof, are contemplated in the compounds of the present invention.

### DETAILED DESCRIPTION OF THE INVENTION

I. Introduction

The present invention relates to benzo[b]thiophenes of Formulas I-IVc, R³, R⁴, R⁵, R⁶, R⁷, Y, and L have any of the values defined therefor in the specification, and pharmaceutically acceptable salts thereof, that are useful as agents in the treatment of diseases and conditions, including inflammatory diseases, cardiovascular diseases, and cancers. Also provided are pharmaceutical compositions comprising one or more compounds of Formulas I-IVc.

II. Preparation of Compounds

Compounds of the present invention (e.g., compounds of Formulas I-IVc) can be prepared by applying synthetic methodology known in the art and synthetic methodology outlined in the schemes set forth below.

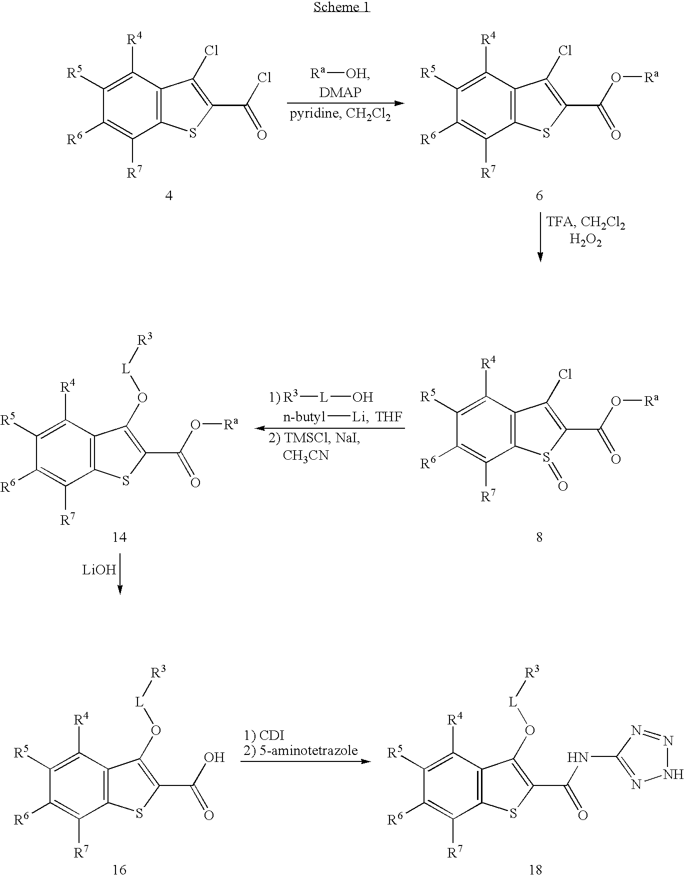

In Scheme 1, an acid chloride 4 (e.g., 3-chloro-6-fluoro-5-methoxy-benzo[b]thiophene-2-carbonyl chloride) is reacted with R^{a}—OH (e.g., phenol, isopropyl alcohol, methanol, etc.), pyridine or triethylamine (TEA), and 4-dimethylaminopyridine (DMAP) in CH₂Cl₂ to yield the ester 6 (e.g., 3-chloro-6-fluoro-5-methoxy-benzo[b]thiophene-2-carboxylic acid isopropyl ester). R^{a}—OH can be any suitable alcohol, where R^{a} is a C₁-C₄ alkyl, phenyl, methyl, isopropyl, etc., that protects the carboxyl group and can be removed subsequently by base hydrolysis. Acid chlorides of formula 4 can be synthesized using methods that are well-known in the art (see e.g., Pakray and Castle (1986) J. Heterocyclic Chem. 23: 1571-1577; Boschelli et al. (1995) J. Med. Chem. 38: 4597-4614; Connor et al. (1992) J. Med. Chem. 35: 958-965).

The ester 6 is then oxidized to the 1-oxo-benzo[b]thiophene compound 8 (e.g., 3-chloro-6-fluoro-5-methoxy-1-oxo-1H-1λ⁴-benzo[b]thiophene-2-carboxylic acid isopropyl ester) using trifluroacetic acid (TFA), CH₂Cl₂, and hydrogen peroxide (H₂O₂). A solution of an alkyl lithium (e.g., n-butyllithium) treated 10 (R³-L-OH) in THF is then added to a solution of 8 in THF to yield the 3-substituted benzo[b]thiophene 12 (e.g., 3-cyclohexyloxy-6-fluoro-5-methoxy-1-oxo-1H-1λ⁴-benzo[b]thiophene-2-carboxylic acid isopropyl ester). Alternatively, sodium hydride treated 10 (R³-L-OH) can be added to a heterogeneous mixture of 8 in dioxane to yield 12. R³ and L are as defined herein. A variety of R³-L-OH compounds can be used including but not limited to, tetrahydro-4H-pyran-4-ol, cyclopentanol, cyclohexyl-methanol, (3,5-dimethyl-cyclohexyl)-methanol, phenol, biphenyl-3-ol, 3-methyl-phenol, 3-nitro-phenol, 3-acetylamino-phenol, naphthalen-2-ol, 3-ethyl-phenol, 3-morpholin-4-yl-phenol, 3-isopropyl-phenol, 3-isopropyl-5-methyl-phenol, 2-ethyl-phenol, 4-cyclohexyl-phenol, and phenyl-methanol.

12 in acetonitrile is then treated with sodium iodide (NaI) followed by chlorotrimethylsilane (TMSCl) to provide 14 (e.g., 3-cyclohexyloxy-6-fluoro-5-methoxy-benzo[b]thiophene-2-carboxylic acid isopropyl ester). 14 is then saponified with an inorganic base such as LiOH or NaOH in a solution of MeOH and THF; dioxane and water; or methanol and water, to provide 16 (e.g., 3-cyclohexyloxy-6-fluoro-5-methoxy-benzo[b]thiophene-2-carboxylic acid). The carboxylic acid 16 is then treated with carbonyl diimidazole (CDI) in a non-protic solvent such as THF (tetrahydrofuran), followed by the addition of a 5-aminotetrazole to provide the carboxamide 18 (e.g., 3-cyclohexyloxy-6-fluoro-5-methoxy-benzo[b]thiophene-2-carboxylic acid (2H-tetrazol-5-yl)-amide).

Alternatively, 16 in anhydrous CH₂Cl₂ can be treated with a catalytic amount of DMF followed by oxalyl chloride. Acetonitrile is then added to this mixture, followed by the addition of 5-aminotetrazole and triethylamine to give 18.

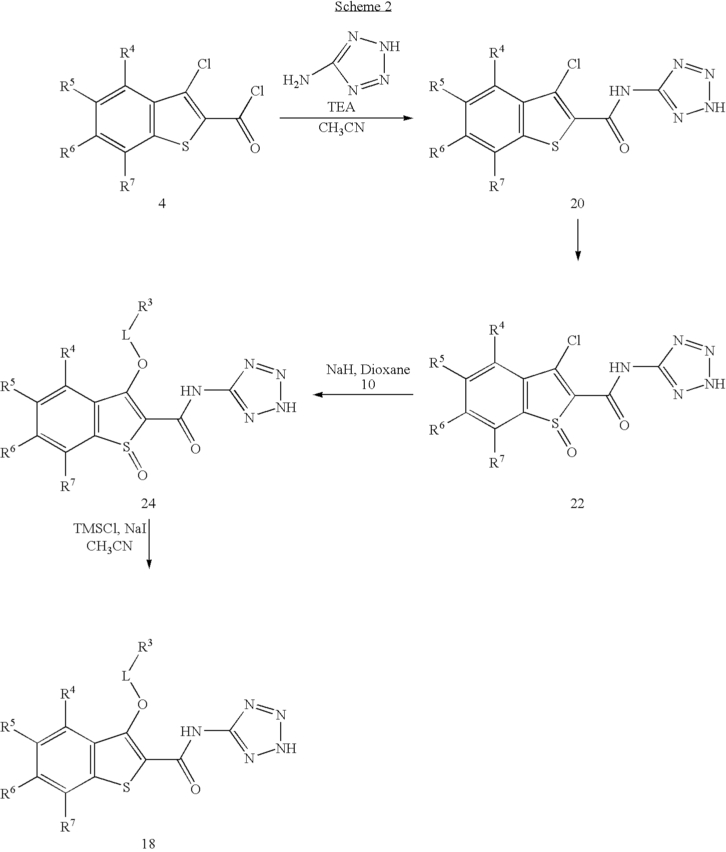

In Scheme 2, a compound 4 (e.g., 3-chloro-6-fluoro-5-methoxy-benzo[b]thiophene-2-carbonyl chloride) is refluxed with 5-aminotetrazole and triethylamine (TEA) in acetonitrile (CH₃CN) to provide 20 (e.g., 3-chloro-6-fluoro-5-methoxy-benzo[b]thiophene-2-carboxylic acid (2H-tetrazol-5-yl)-amide). 20 is then oxidized using aqueous H₂O₂ in CH₂Cl₂ and trifluoroacetic acid (TFA) to give 22 (e.g., 3-chloro-6-fluoro-5-methoxy-1-oxo-1h-1λ⁴-benzo[b]thiophene-2-carboxylic acid (2H-tetrazol-5-yl)-amide).

22 is mixed with 10 in dioxane and then reacted with 2 equivalents of NaH to give 24 (e.g., 3-(4-cyclopentyloxy-phenoxy)-6-fluoro-5-methoxy-1-oxo-1h-1λ⁴-benzo[b]thiophene-2-carboxylic acid (2H-tetrazol-5-yl)-amide). 24 is then treated with sodium iodide in acetonitrile followed by the addition of chlorotrimethylsilane (TMSCl) to give 18 (e.g., 3-(4-cyclopentyloxy-phenoxy)-6-fluoro-5-methoxy-benzo[b]thiophene-2-carboxylic acid (2H-tetrazol-5-yl)-amide).

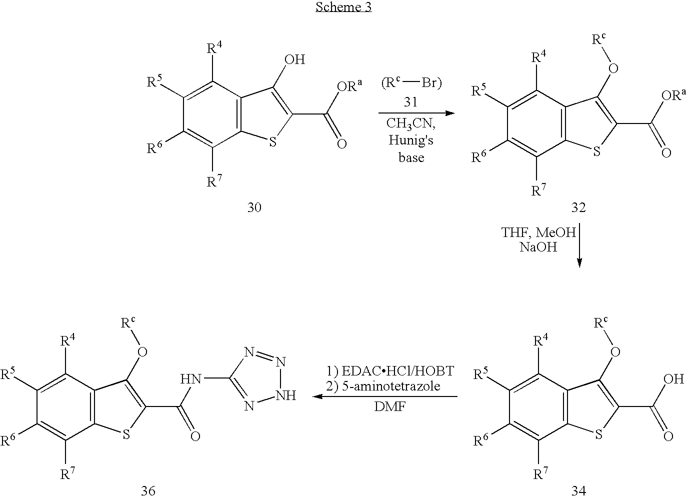

In Scheme 3, 30 (e.g., 6-fluoro-3-hydroxy-5-methoxy-benzo[b]thiophene-2-carboxylic acid isopropyl ester) in acetonitrile is treated with di-isopropyl ethyl amine (Hünig's base) followed by 31 (e.g., 3-bromo benzyl bromide) to provide 32 (e.g., 3-benzyloxy-6-fluoro-5-methoxy-benzo[b]thiophene-2-carboxylic acid isopropyl ester). Examples of 31 (R^{c}—Br) include, but are not limited to, 3,4-difluoro-benzylbromide, 4-bromomethyl-biphenyl, 1-bromomethyl-3-trifluoromethyl-benzene, 1-bromomethyl-3,5-dimethoxy-benzene, 1-bromomethyl-4-tert-butyl-benzene, 2-bromomethyl-1,3,4-trifluoro-benzene, and 2-bromomethyl-naphthalene. R^{c}—Br is a compound of formula R³-L-Br, where L is present (e.g., a C₁-C₄ alkylene) if R³ is a substituted or unsubstituted phenyl.

32 is then saponified with an inorganic base as described in Scheme 1 to provide the carboxylic acid 34 (e.g., 3-benzyloxy-6-fluoro-5-methoxy-benzo[b]thiophene-2-carboxylic acid). 34 in DMF is then treated with EDAC.HCl (N-(3-dimethylaminopropyl)-N′-ethylcarbodiimide hydrochloride), HOBT (1-hydroxy-6-(trifluoromethyl)benzotriazole) and 5-aminotetrazole to provide the carboxamide 36 (e.g., 3-benzyloxy-6-fluoro-5-methoxy-benzo[b]thiophene-2-carboxylic acid (2H-tetrazol-5-yl)-amide).

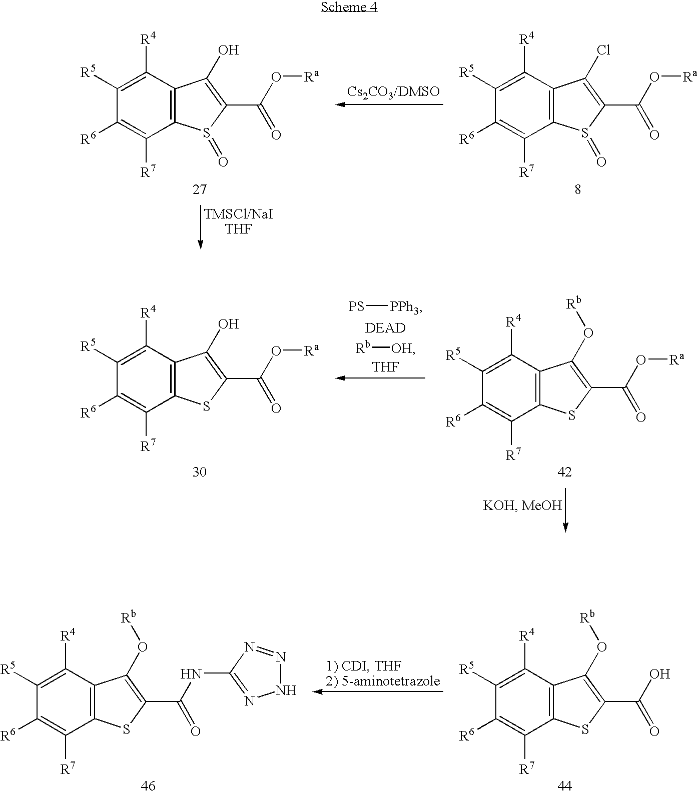

In Scheme 4, 8 is treated with Cs₂CO₃ in DMSO or LiOH in water/dioxane (1:1) to generate 29. 29 is then reduced to 30 as in the transformation of 24 to 18 in Scheme 2. PS-triphenylphosphine (polystyrene-triphenylphosphine) or triphenylphosphine is added to a solution of 30 (e.g., 6-fluoro-3-hydroxy-5-methoxy-benzo[b]thiophene-2-carboxylic acid isopropyl ester) in THF under nitrogen gas. Diethyl azodicarboxylate (DEAD) is added, followed by the addition of R^{b}—OH to yield 42 (e.g., 3-cyclohexylmethoxy-6-fluoro-5-methoxy-benzo[b]thiophene-2-carboxylic acid isopropyl ester). R^{b}—OH is a compound of formula R³-L-OH, where L is present if R³ is a substituted or unsubstituted phenyl group. Examples of R^{b}—OH include, but are not limited to, 2-cyclopropyl-ethanol, (2,2-dichloro-cyclopropyl)-methanol, cyclohexyl-methanol, and tetrahydro-furan-3-ol.

The ester 42 in methanol is hydrolyzed using an inorganic base, such as potassium hydroxide, to yield the corresponding carboxylic acid 44 (e.g., 3-cyclohexylmethoxy-6-fluoro-5-methoxy-benzo[b]thiophene-2-carboxylic acid). 44 is converted to the carboxamide 46 (e.g., 3-cyclohexylmethoxy-6-fluoro-5-methoxy-benzo[b]thiophene-2-carboxylic acid (2H-tetrazol-5-yl)-amide) in an analogous manner to the transformation of 16 to 18 in Scheme 1.

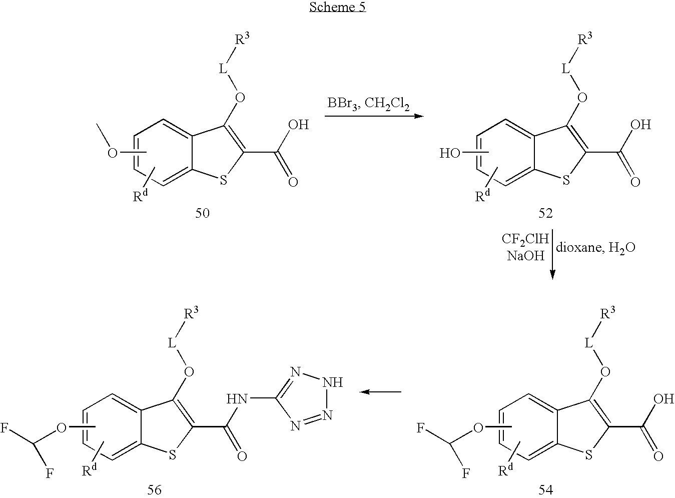

In Scheme 5, the benzo[b]thiophene 50 (e.g., 3-cyclohexyloxy-6-fluoro-5-methoxy-benzo[b]thiophene-2-carboxylic acid) is converted to the corresponding hydroxy-benzo[b]thiophene 52 (e.g., 3-cyclohexyloxy-6-fluoro-5-hydroxy-benzo[b]thiophene-2-carboxylic acid) using boron tribromide in anhydrous CH₂Cl₂. R^{d} is F, Br, I, or Cl.

The difluoromethyl-benzo[b]thiophene 54 can be provided from 52. The treatment of 52 with an inorganic base (e.g., NaOH) and chlorodifluoromethane (CF₂ClH) in dioxane and water yields 54 (e.g., 3-cyclohexyloxy-5-difluoromethoxy-6-fluoro-benzo[b]thiophene-2-carboxylic acid). 54 is then coupled with 5-aminotetrazole as described in Scheme 1 to give the corresponding aminotetrazole derivative 56 (e.g., 3-cyclohexyloxy-5-difluoromethoxy-6-fluoro-benzo[b]thiophene-2-carboxylic acid (2H-tetrazol-5-yl)-amide).

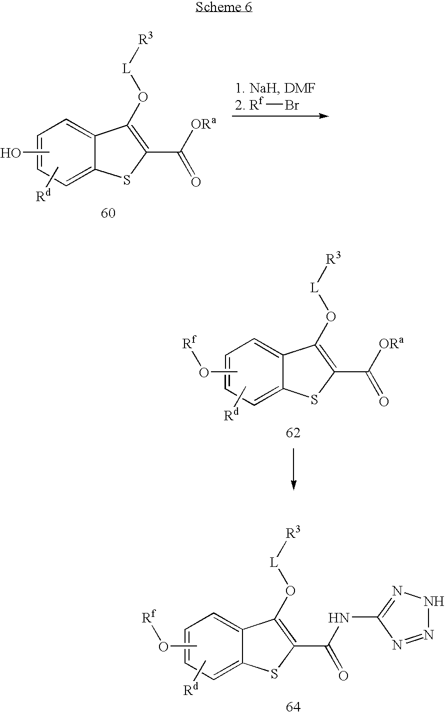

In Scheme 6, the hydroxy-benzo[b]thiophene 60 (e.g., 3-cyclohexyloxy-6-fluoro-5-hydroxy-benzo[b]thiophene-2-carboxylic acid isopropyl ester) is reacted with a base such as sodium hydride in dimethylformamide, followed by the addition of R^{f}—Br (e.g., bromo-ethane, 2-bromo-1,1,1-trifluoro-ethane, etc.), where R^{f} is a C₁-C₃alkyl group (e.g., methyl, ethyl, propyl, isopropyl, 2,2,2-trifluoro-ethyl, etc.) to provide the C₁-C₃-alkoxy substituted benzothiophene 62 (e.g., 3-cyclohexyloxy-5-ethoxy-6-fluoro-benzo[b]thiophene-2-carboxylic acid isopropyl ester). 62 is then saponified and coupled with 5-aminotetrazole as described in Scheme 1 to give the corresponding aminotetrazole derivative 64 (e.g., 3-cyclohexyloxy-5-ethoxy-6-fluoro-benzo[b]thiophene-2-carboxylic acid (2H-tetrazol-5-yl)-amide).

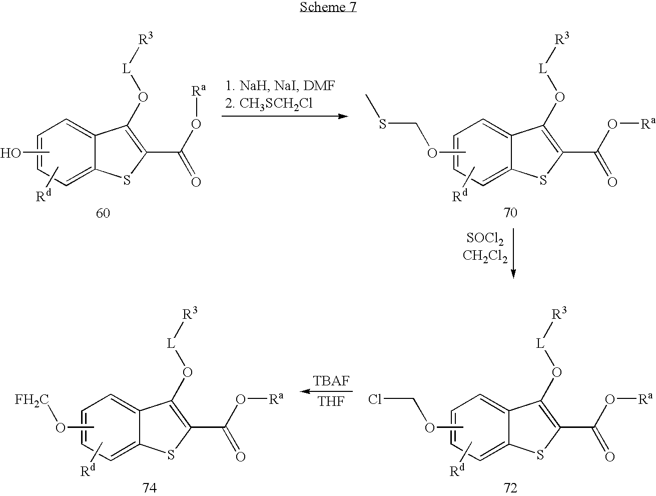

In Scheme 7, the benzothiophene 60 is reacted in series of reactions analogous to those described in Ringom and Benneche (1999) Acta Chemica Scandinavica 53(1): 41-47 to provide 74. First, hydroxy substituted benzothiophene 60 (e.g., 3-(3,5-Dimethyl-cyclohexyloxy)-6-fluoro-5-hydroxy-benzo[b]thiophene-2-carboxylic acid isopropyl ester) is reacted with NaI and a hydride base such as NaH, in dimethylformamide (DMF), followed by the addition of chloro-methylsulfanyl-methane (CH₃SCH₂Cl) to provide the methylthiomethylether substituted benzothiophene 70 (e.g., 3-(3,5-Dimethyl-cyclohexyloxy)-6-fluoro-5-methylsulfanylmethoxy-benzo[b]thiophene-2-carboxylic acid isopropyl ester). The methylthiomethylether is then transformed into a chloromethoxy group by reacting 70 with sulfonyl chloride in a solvent such as dichloromethane to yield 72 (e.g., 5-chloromethoxy-3-(3,5-dimethyl-cyclohexyloxy)-6-fluoro-benzo[b]thiophene-2-carboxylic acid isopropyl ester). The chloromethoxy group of 72 is reacted with a reagent such as TBAF (tetrabutylammonium fluoride) to provide the fluoromethoxy substituted compound 74 (e.g., 3-(3,5-dimethyl-cyclohexyloxy)-6-fluoro-5-fluoromethoxy-benzo[b]thiophene-2-carboxylic acid isopropyl ester). 74 is then hydrolyzed with base and reacted with 5-aminotetrazole as described in Scheme 1 to provide the corresponding benzo[b]thiophene-2-carboxylic acid (2H-tetrazol-5-yl)-amide (e.g., 3-(3,5-dimethyl-cyclohexyloxy)-6-fluoro-5-fluoromethoxy-benzo[b]thiophene-2-carboxylic acid (2H-tetrazol-5-yl)-amide).

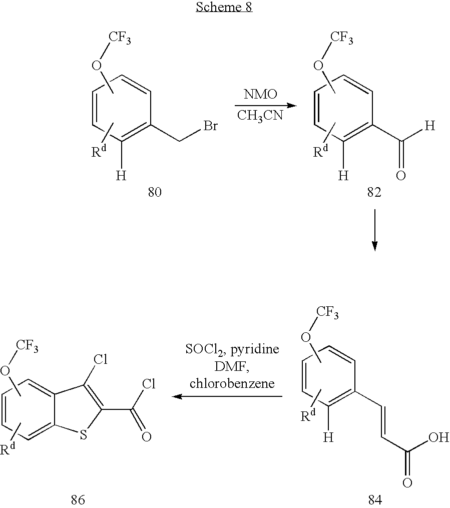

In Scheme 8, the bromomethyl group of 80 (e.g., 4-bromomethyl-1-fluoro-2-trifluoromethoxy-benzene; EP0075146B1) can be oxidized to the aldehyde 82 (e.g., 4-fluoro-3-trifluoromethoxy-benzaldehyde) using an oxidant such as N-methylmorpholine N-oxide (NMO) in a solvent such as acetonitrile. The use of NMO to oxidize activated halides is known in the art (see e.g., Griffith et al. (1992) Synth. Communications 22(13): 1967-1971).

82 is then refluxed with malonic acid in a mixture of piperidine and pyridine to yield the acrylic acid 84 (e.g., 3-(4-fluoro-3-trifluoromethoxy-phenyl)-acrylic acid). 84 is then reacted with thionyl choride in a mixture of pyridine, dimethylformamide (DMF), and chlorobenzene under reflux conditions to yield 86 (e.g., 3-chloro-6-fluoro-5-trifluoromethoxy-benzo[b]thiophene-2-carbonyl chloride). 86 can then be reacted in a similar manner to that described for Scheme 1 to form a compound of formula 18.

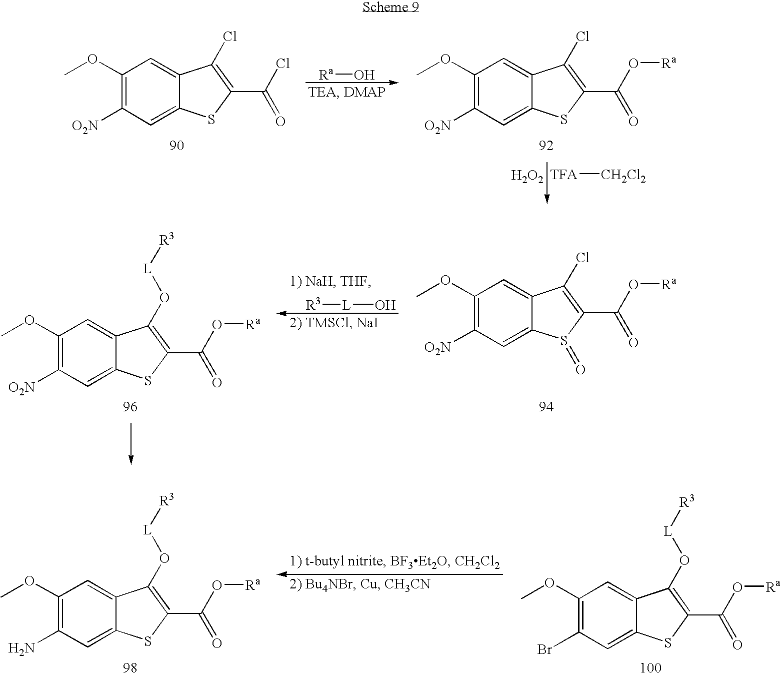

In Scheme 9, the nitro-benzothiophene 90 (e.g., 3-chloro-5-methoxy-6-nitro-benzo[b]thiophene-2-carbonyl chloride) is reacted with DMAP (4-(dimethylamino)pyridine), triethyl amine, and an alcohol R^{a}—OH (e.g., isopropanol, phenol, methanol, etc.) to provide the ester 92 (e.g., 3-chloro-5-methoxy-6-nitro-benzo[b]thiophene-2-carboxylic acid isopropyl ester). 92 in TFA (trifluoroacetic acid) and CH₂Cl₂ is oxidized with H₂O₂ to yield 94 (e.g., 3-chloro-5-methoxy-6-nitro-1-oxo-benzo[b]thiophene-2-carboxylic acid isopropyl ester). 94 is reacted with a compound of formula R³-L-OH (e.g., 4-cyclohexyl-phenol) and NaH in anhydrous THF, followed by that addition of chlorotrimethylsilane and NaI to generate 96 (e.g., 3-(4-cyclohexyl-phenoxy)-5-methoxy-6-nitro-benzo[b]thiophene-2-carboxylic acid isopropyl ester).

96 is then reduced using Rainey Nickel under high pressure hydrogen gas in a Parr reaction vessel to provide the amino-benzothiophene 98 (e.g., 6-amino-3-(4-cyclohexyl-phenoxy)-5-methoxy-benzo[b]thiophene-2-carboxylic acid isopropyl ester).

98 in anhydrous CH₂Cl₂ is first reacted with BF₃.Et₂O (boron fluoride-diethyl etherate) and then a solution of t-butyl nitrite in CH₂Cl₂. The reaction is worked up and the resulting residue is dissolved in anhydrous CH₃CN and transferred to a solution of tetrabutylammonium bromide (Bu₄NBr) in CH₃CN. The reaction is stirred and then copper was added to generate 100 (e.g., 6-bromo-3-(4-cyclohexyl-phenoxy)-5-methoxy-benzo[b]thiophene-2-carboxylic acid isopropyl ester).

100 is then saponified and coupled with 5-aminotetrazole in a manner as described in Scheme 1 to provide for the corresponding benzo[b]thiophene-2-carboxylic acid (2H-tetrazol-5-yl)-amide (e.g., 6-bromo-3-(4-cyclohexyl-phenoxy)-5-methoxy-benzo[b]thiophene-2-carboxylic acid (2H-tetrazol-5-yl)-amide). Alternatively, tetrabutylammonium chloride can be substituted for tetrabutylammonium bromide in Scheme 9 to provide the corresponding chloro-substituted benzothiophene (e.g., 6-chloro-3-(4-cyclohexyl-phenoxy)-5-methoxy-benzo[b]thiophene-2-carboxylic acid (2H-tetrazol-5-yl)-amide).

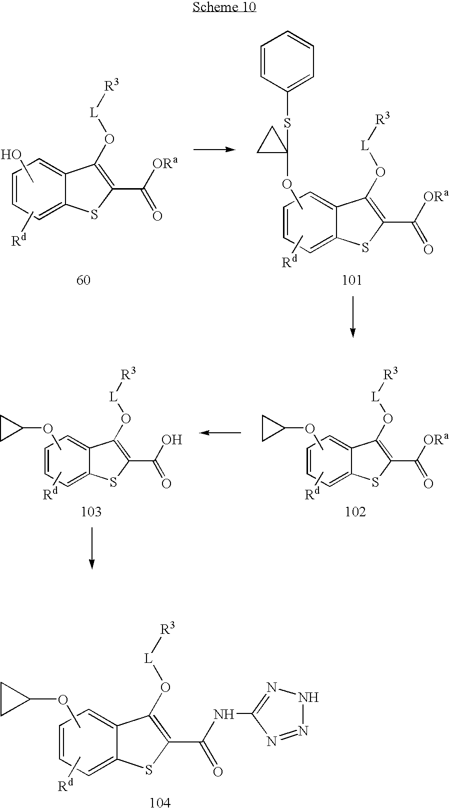

In Scheme 10, the cyclopropyloxy substituted benzo[b]thiophene 116 can be generated as depicted. The 5-hydroxy-benzo[b]thiophene 60 can be subjected to a cyclopropanation procedure known in the art such as those described in U.S. Pat. No. 6,515,126. The benzothiophene 60 can be reacted with (1-iodo-cycloprop-1-yl)phenylsulfide and a base such as silver carbonate to yield 101. 101 can then be reacted with a metal naphthalenide (e.g., lithium naphthalenide) in an aprotic solvent such as THF or ether at a temperature of around −80° C. to yield a compound of formula 102. Alternatively, the phenylthio group of 101 can be oxidized with a reagent such as ozone in the presence of aluminium oxide, in a chlorinated hydrocarbon solvent (e.g., chloroform) at room temperature. The resulting phenylsulfonyl group can be removed with sodium amalgam in the presence of disodium hydrogen orthophosphate, in an alcohol solvent such as methanol to yield 102. The ester 102 is then saponified as described in Scheme 1 to the acid 103. 103 can be coupled to 5-aminotetrazole as described in Scheme 1 to yield 104.

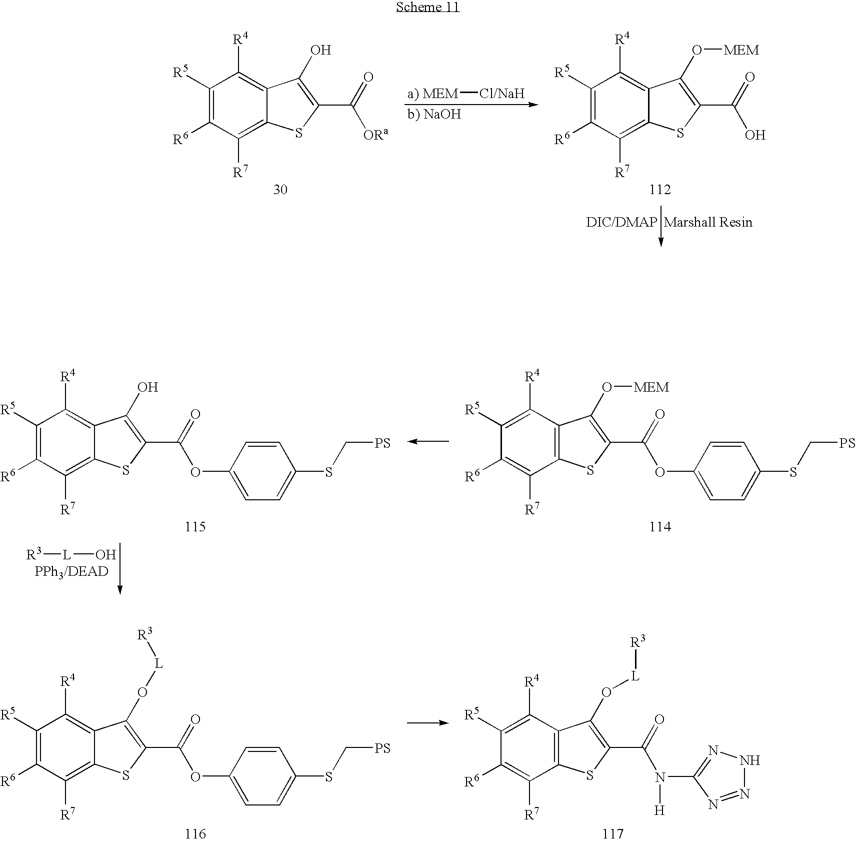

In Scheme 11, a solid phase synthesis of compound of formula 116 is depicted. A solution of 30 (e.g., 6-fluoro-3-hydroxy-5-methoxy-benzo[b]thiophene-2-carboxylic acid methyl ester) in a solvent such as DMF is treated with a hydride such as potassium hydride or sodium hydride followed by the addition of a suitable hydroxyl protecting group reagent such as MEM-Cl (2-methoxyethoxymethyl chloride; CH₃OCH₂CH₂OCH₂—Cl) to give compound 111 (e.g., 6-fluoro-5-methoxy-3-(2-methoxy-ethoxymethoxy)-benzo[b]thiophene-2-carboxylic acid methyl ester). Those of skill in the art will recognize that other hydroxyl protecting groups in addition to the 2-methoxyethoxymethyl group can be used in Scheme 3 (see e.g., Greene and Wuts, Protective Groups in Organic Synthesis, 2nd ed., Chapter 2 (John Wiley & Sons, Inc., 1991)). The ester 111 in THF and water is then hydrolyzed with a base such as NaOH to provide the carboxylic acid 112 (e.g., 6-fluoro-5-methoxy-3-(2-methoxy-ethoxymethoxy)-benzo[b]thiophene-2-carboxylic acid).

112 in dichloromethane is then conjugated to a solid phase resin such as Marshall resin by reaction with di-isopropyl carbodiimide (DIC) or dicyclohexylcarbodiimide, and Marshall resin (phenol sulfide polystyrene (PS) resin; Marshall and Liener (1970) J. Org. Chem. 35: 867-868) to yield 114. The 2-methoxy-ethoxymethoxy group is then hydrolyzed from 114 in dichloromethane using a suitable acid such as triflouroacetic acid to yield the polymer supported alcohol 115 (e.g., 6-Fluoro-3-hydroxy-5-methoxy-benzo[b]thiophene-2-carboxylic acid-polymer supported). 115 in dichloromethane is combined with a solution of triphenylphosphine and diethylazidodicarboxylate treated R³-L-OH, where L is present if R³ is a substituted or unsubstituted phenyl, to yield the R³-L- substituted compound 116. 116 is then coupled to 5-amino-tetrazole as described in Scheme 1 to yield 117.

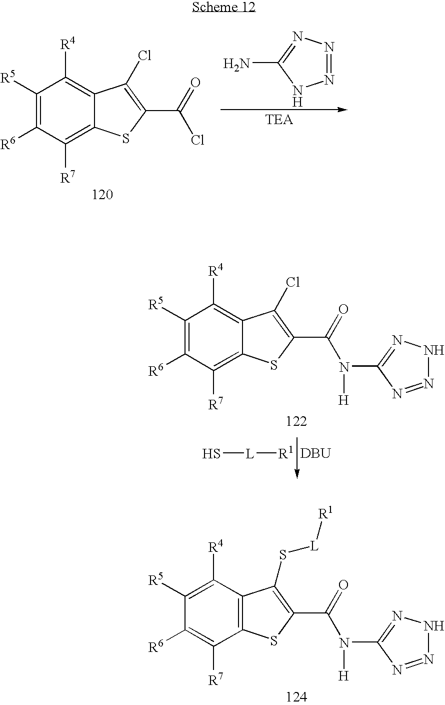

In Scheme 12, the acid chloride 120 is coupled to 5-aminotetrazole in the presence of a base such as triethylamine in a suitable solvent such as acetonitrile to yield 122. 122 is then coupled to a thiol 123, HS-L-R³, at the 3-position by treatment with a tertiary amine such as 1,8-diazabicyclo[5.4.0]undec-7-ene (DBU) or 1,5-diazabicyclo[4.3.0]non-5-ene (DBN) to provide 124. Examples of compound of formula 123 include, but are not limited to benzenethiol, 4-mercapto-phenol, 2-methyl-benzene thiol, 2-phenyl-ethanethiol, and 3-(4-mercapto-phenyl)-propionic acid methyl ester. In certain embodiments, an ester substituent of R³ on 124 can be hydrolyzed to the corresponding acid using a suitable base such as sodium hydroxide in a solvent such as tetrahydrofuran (THF).

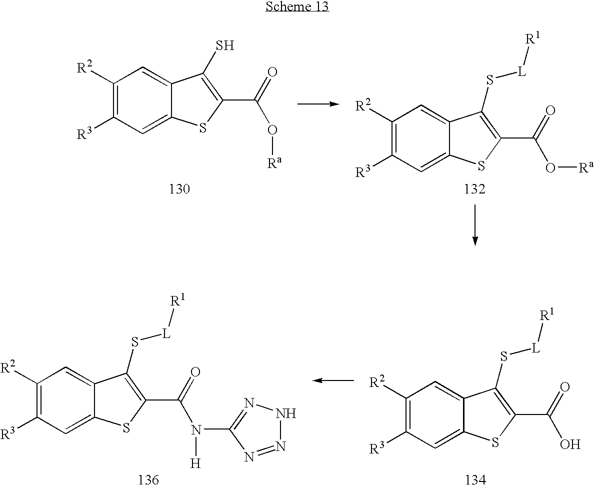

In Scheme 13, the benzothiophene 130 (e.g., 3-mercapto-5,6-dimethoxy-benzo[b]thiophene-2-carboxylic acid benzyl ester) is reacted with a base such as triethylamine and a compound of formula 131 (X—C₁-C₄alkylene-R¹) (e.g., 4-bromomethyl-benzonitrile), where X is a halo group, in a solvent such as acetonitrile to give 132 (e.g., 3-(4-cyano-benzylsulfanyl)-5,6-dimethoxy-benzo[b]thiophene-2-carboxylic acid benzyl ester). R^{a}—OH can be any suitable alcohol (e.g., phenol, isopropyl alcohol, phenyl-methanol, methanol, etc.), where R^{a} is a C₁-C₄ alkyl, isopropyl, phenyl, benzyl, methyl, etc., that protects the carboxylic acid group and can be removed subsequently by base hydrolysis.

The ester 132 is then hydrolyzed to 134 using a base such as LiOH, KOH, or NaOH in a solvent such as a mixture of THF and methanol. The carboxylic acid 134 (e.g., 3-(4-cyano-benzylsulfanyl)-5,6-dimethoxy-benzo[b]thiophene-2-carboxylic acid) in anhydrous CH₂Cl₂ or can be treated with a catalytic amount of DMF (dimethylformamide) followed by oxalyl chloride. Acetonitrile is then added to this mixture, followed by the addition of 5-aminotetrazole and triethylamine to give 136 (e.g., 3-(4-cyano-benzylsulfanyl)-5,6-dimethoxy-benzo[b]thiophene-2-carboxylic acid (1H-tetrazol-5-yl)-amide). The reaction of 134 with oxalyl chloride and DMF can be carried out in THF (tetrahydrofuran).

Alternatively, the acid 134 can be reacted with carbonyl diimidazole (CDI) in an aprotic solvent such as THF (tetrahydrofuran), followed by the addition of a 5-aminotetrazole to provide the carboxamide 136.

III. Evaluation of Compounds

Compounds of the present invention (e.g., compounds of Formulas I-IVc and pharmaceutically acceptable salts thereof) can be assayed for their ability to inhibit a PI3K. Examples of these assays are set out below and include in vitro and in vivo assays of PI3K activity.

In certain embodiments of the present invention are compounds that selectively inhibit one or more PI3Ks as compared to one or more enzymes including, but not limited to, a cyclic nucleotide dependent protein kinase, PDGF, a tyrosine kinase, a MAP kinase, a MAP kinase kinase, a MEKK, a cyclin-dependent protein kinase. In other embodiments of the invention are compounds that selectively inhibit one PI3K as compared to another PI3K. For example, in certain embodiments, compounds of the present invention display the ability to selectively inhibit PI3Kγ as compared to PI3Kα or PI3Kβ. A compound selectively inhibits a first enzyme as compared to a second enzyme, when the IC₅₀ of the compound towards the first enzyme is less than the IC₅₀ of the compound towards the second compound. The IC₅₀ can be measured, for example, in an in vitro PI3K assay.

In presently preferred embodiments, compounds of the present invention can be assessed for their ability to inhibit PI3Kactivity in an in vitro or an in vivo assay (see below).

PI3K assays are carried out in the presence or absence of a PI3K inhibitory compound, and the amount of enzyme activity is compared for a determination of inhibitory activity of the PI3K inhibitory compound.

Samples that do not contain a PI3K inhibitory compound are assigned a relative PI3K activity value of 100. Inhibition of PI3K activity is achieved when the PI3K activity in the presence of a PI3K inhibitory compound is less than the control sample (i.e., no inhibitory compound). The IC₅₀ of a compound is the concentration of compound that exhibits 50% of the control sample activity. In certain embodiments, compounds of the present invention have an IC₅₀ of less than about 100 μM. In other embodiments, compounds of the present invention have an IC₅₀ of about 1 μM or less. In still other embodiments, compounds of the present invention have an IC₅₀ of about 200 nM or less.

PI3Kγ assays have been described in the art (see e.g., Leopoldt et al. J. Biol. Chem., 1998;273:7024-7029). Typically, a sample containing a complex of p101 and p110γ protein are combined with Gβ and Gγ proteins (e.g., G protein β₁/γ₂ subunits). Radiolabeled ATP (e.g., γ-³²P-ATP) is then added to this mixture. The lipid substrates are formed by creating PIP₂ containing lipid micelles. The reactions are then started by adding the lipid and enzyme mixtures and are stopped with the addition of H₃PO₄. The lipid products are then transferred to a glass fiber filter plate, and washed with H₃PO₄ several times. The presence of radioactive lipid product (PIP₃) can be measured using radiometric methods that are well-known in the art.

The activity of growth factor regulated PI3Ks can also be measured using a lipid kinase assay. For example, PI3Kα can be assayed using samples that contain a regulatory and a catalytic subunit. An activating peptide (e.g., pY peptide, SynPep Corp.) is added to the sample with radiolabeled ATP. PIP₂ containing lipid micelles are then added to the sample to start the reaction. The reactions are worked up and analyzed as described for the PI3Kγ assay just described. Assays can also be carried out using cellular extracts (Susa et al. J. Biol. Chem., 1992;267:22951-22956).

IV. Pharmaceutically Acceptable Salts and Solvents

The compounds to be used in the present invention can exist in unsolvated forms as well as solvated forms, including hydrated forms. In general, the solvated forms, including hydrated forms, are equivalent to unsolvated forms and are intended to be encompassed within the scope of the present invention.

The compounds of the present invention (e.g., compounds of Formulas I-IVc) are capable of further forming both pharmaceutically acceptable salts, including but not limited to acid addition and/or base salts. Pharmaceutically acceptable salts of the compounds of formula (I) include the acid addition and base salts (including disalts) thereof. Examples of suitable salts can be found for example in Stahl and Wermuth, Handbook of Pharmaceutical Salts: Properties, Selection, and Use, Wiley-VCH, Weinheim, Germany (2002); and Berge et al., “Pharmaceutical Salts,” J. of Pharmaceutical Science, 1977;66:1-19.

Pharmaceutically acceptable acid addition salts of the compounds of Formulas I-IVc include non-toxic salts derived from inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include the acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate (methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), and xinafoate salts, and the like of compounds of Formulas I-IVc. Also contemplated are the salts of amino acids such as arginate, gluconate, galacturonate, and the like.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are aluminium, calcium, magnesium, potassium, sodium, and the like. Examples of suitable amines include arginine, choline, chloroprocaine, N,N′-dibenzylethylenediamine, diethylamine, diethanolamine, diolamine, ethylenediamine (ethane-1,2-diamine), glycine, lysine, meglumine, N-methylglucamine, olamine, procaine (benzathine), and tromethamine.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

V. Pharmaceutical Compositions and Methods of Administration

This invention also provides for pharmaceutical compositions comprising a therapeutically effective amount of a compound of Formulas I-IVc, or a pharmaceutically acceptable salt thereof together with a pharmaceutically acceptable carrier, diluent, or excipient therefor. The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disorder or condition being treated when administered alone or in conjunction with another pharmaceutical agent or treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that determination of proper dosage forms, dosage amounts, and routes of administration is within the level of ordinary skill in the pharmaceutical and medical arts, and is described below.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or a disease indicia associated with a PI3K-mediated disorder as measured quantitatively or qualitatively.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep, and cows.

The dose will be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present invention can be administered at a rate determined by factors that can include, but are not limited to, the LD₅₀ of the compound, the pharmacokinetic profile of the compound, contraindicated drugs, and the side-effects of the compound at various concentrations, as applied to the mass and overall health of the subject. Administration can be accomplished via single or divided doses.

Examples of a typical tablet, parenteral, and patch formulation include the following:

### Tablet Formulation Example 1

Tablet Formulation

| Ingredient | Amount | |
|---|---|---|
| Compound of Formulas I-IVc | 50 | mg |
| Lactose | 80 | mg |
| Cornstarch (for mix) | 10 | mg |
| Cornstarch (for paste) | 8 | mg |
| Magnesium Stearate (1%) | 2 | mg |
| | 150 | mg |

The compounds of the present invention (e.g., a compound of Formulas I-IVc, or a pharmaceutically acceptable salt thereof) can be mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for treatment of a PI3K-mediated disorder or condition.

### Parental Solution Formulation Example 1

In a solution of 700 mL of propylene glycol and 200 mL of water for injection can be added 20.0 g of a compound of the present invention. The mixture is stirred, and the pH is adjusted to 5.5 with hydrochloric acid. The volume is adjusted to 1000 mL with water for injection. The solution is sterilized, filled into 5.0 mL ampules, each containing 2.0 mL (40 mg of invention compound), and sealed under nitrogen. The solution is administered by injection to a subject suffering from a PI3K-mediated disorder or condition and in need of treatment.

### Patch Formulation Example 1

Ten milligrams of a compound of the present invention can be mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of a PI3K-mediated disorder or condition.

V. Methods for Treating PI3K-Mediated Disorders and Conditions

The compounds of the present invention and pharmaceutical compositions comprising a compound of the present invention can be administered to a subject suffering from a PI3K-mediated disorder or condition. PI3K-mediated disorders and conditions can be treated prophylactically, acutely, and chronically using compounds of the present invention, depending on the nature of the disorder or condition. Typically, the host or subject in each of these methods is human, although other mammals can also benefit from the administration of a compound of the present invention.

In therapeutic applications, the compounds of the present invention can be prepared and administered in a wide variety of oral and parenteral dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. Also, the compounds described herein can be administered by inhalation, for example, intranasally. Additionally, the compounds of the present invention can be administered transdermally, topically, via implantation, transdermally, topically, and via implantation. In certain embodiments, the compounds of the present invention are delivered orally. The compounds can also be delivered rectally, bucally, intravaginally, ocularly, andially, or by insufflation.

The compounds utilized in the pharmaceutical method of the invention can be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired. The term “treatment” includes the acute, chronic, or prophylactic diminishment or alleviation of at least one symptom or characteristic associated with or caused by the disorder being treated. For example, treatment can include diminishment of several symptoms of a disorder, inhibition of the pathological progression of a disorder, or complete eradication of a disorder. The compounds of the present invention can be co-administered to a subject. The term “co-administered” means the adminstration of two or more different pharmaceutical agents or treatments (e.g., radiation treatment) that are administered to a subject by combination in the same pharmacetical composition or separate pharamaceutical compositions. Thus co-adminstration involves adminstration at the same time of a single pharmaceutical composition comprising two or more pharmaceutical agents or administration of two or more different compositions to the same subject at the same or different times. For example, a subject that is administered a first dosage that comprises a compound of the present invention at 8 a.m. and then is adminstred CELEBREX® at 1-12 hours later, e.g., 6 p.m., of that same day has been co-administered with a compound of the present invention and CELEBREX®. Alternatively, for example, a subject could be administred with a single dosage comprising a compound of the present invention and CELEBREX® at 8 a.m. has been co-administered with a compound of the present invention and CELEBREX®.

Thus, compounds of the invention can also be co-administered with compounds that are useful for the treatment of cancer (e.g., cytotoxic drugs such as TAXOL®, taxotere, GLEEVEC® (Imatinib Mesylate), adriamycin, daunomycin, cisplatin, etoposide, a vinca alkaloid, vinblastine, vincristine, methotrexate, or adriamycin, daunomycin, cis-platinum, etoposide, and alkaloids, such as vincristine, farnesyl transferase inhibitors, endostatin and angiostatin, VEGF inhibitors, and antimetabolites such as methotrexate. The compounds of the present invention may also be used in combination with a taxane derivative, a platinum coordination complex, a nucleoside analog, an anthracycline, a topoisomerase inhibitor, or an aromatase inhibitor). Radiation treatments can also be co-administered with a compound of the present invention for the treatment of cancers.

The compounds of the invention can also be co-administered with compounds that are useful for the treatment of a thrombolytic disease, heart disease, stroke, etc., (e.g., aspirin, streptokinase, tissue plasminogen activator, urokinase, anticoagulants, antiplatelet drugs (e.g., PLAVIX®; clopidogrel bisulfate), a statin (e.g., LIPITOR® (Atorvastatin calcium), ZOCOR® (Simvastatin), CRESTOR® (Rosuvastatin), etc.), a Beta blocker (e.g, Atenolol), NORVASC® (amlodipine besylate), and an ACE inhibitor (e.g., Accupril® (Quinapril Hydrochloride), Lisinopril, etc.).

The compounds of the invention can also be co-administered for the treatment of hypertension with compounds such as ACE inhibitors, lipid lowering agents such as statins, LIPITOR® (Atorvastatin calcium), calcium channel blockers such as NORVASC® (amlodipine besylate). The compounds of the present invention may also be used in combination with fibrates, beta-blockers, NEPI inhibitors, Angiotensin-2 receptor antagonists and platelet aggregation inhibitors.

For the treatment of inflammatory diseases, including rheumatoid arthritis, the compounds of the invention may be co-administered with agents such as TNF-α inhibitors such as anti-TNFα monoclonal antibodies (such as REMICADE®, CDP-870 and HUMIRA™ (adalimumab) and TNF receptor-immunoglobulin fusion molecules (such as ENBREL®), IL-1 inhibitors, receptor antagonists or soluble IL-1Rα (e.g. KINERET™ or ICE inhibitors), nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin,COX-2 inhibitors (such as CELEBREX® (celecoxib), VIOXX® (rofecoxib), BEXTRA® (valdecoxib), parecoxib, and etoricoxib, metalloprotease inhibitors (preferably MMP-13 selective inhibitors), NEUROTIN®, pregabalin, low dose methotrexate, leflunomide, hydroxychloroquine, d-penicillamine, auranofin or parenteral or oral gold.

The compounds of the invention may be co-administered with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, parecoxicib, rofecoxib and etoricoxib, analgesics and intraarticular therapies such as corticosteroids and hyaluronic acids such as hyalgan and synvisc.

The compounds of the invention may also be co-administered with antiviral agents such as Viracept, AZT, aciclovir and famciclovir, and antisepsis compounds such as Valant.

The compounds of the present invention may further be co-administered with CNS agents such as antidepressants (such as sertraline), anti-Parkinsonian drugs (such as deprenyl, L-Dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), NEURONTIN®, pregabalin, and anti-Alzheimer's drugs such as ARICEPT®, tacrine, propentofylline or metrifonate.

The compounds of the present invention may additionally be co-administered with osteoporosis agents such as EVISTA® (raloxifene hydrochloride) droloxifene, lasofoxifene or fosomax and immunosuppressant agents such as FK-506 and rapamycin.

### EXAMPLES

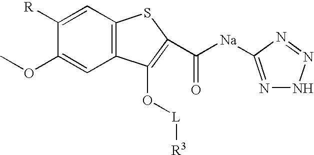

| | | MS | | IC₅₀ |
|---|---|---|---|---|
| Ex. | —L—R³ | (M + 1) | NMR^{a} or Microanalysis | (μm) |
| 1 | 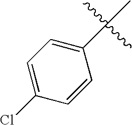 | 420.0 | δ 3.74 (s, 3H) 7.03 (d, J = 9.27 Hz, 2H) 7.07 (d, J = 8.05 Hz, 1H) 7.37 (d, J = 9. Hz, 2H) 8.11 (d,J = 10.98 Hz, 1H) | 0.0765 |
| 2 | 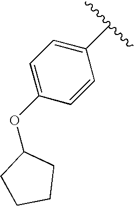 | 470.2 | δ 8.08 (d, J = 10.99 Hz, 1H), 7.01 (d, J = 8.79 Hz, 2H), 6.97 (d, J = 8.06 Hz, 2H), 6.85 (d, J = 9.03 Hz, 1H), 4.71 (m, 1H), 3.69 (s, 3H), 1.83 (m, b, 2H), 1.58 (m, b, 6H) Microanlaysis (C₂₂H₂₀N₅O₄SF): calculated: C = 56.28%, H = 4.29%, N = 14.92%; Found C = 56.19%, H = 4.25%, N = 14.79% | 0.015 |
| 3 | 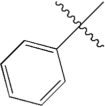 | 386.1 | δ 3.78 (s, 3H), 7.08 (d, J = 8.05 Hz, 1H) 7.16 (m, 3H), 7.43 (m, 2H), 8.18 (d, J = 10.98 Hz, 1H), 11.85 (s, 1H). | 0.0245 |
| 4 | 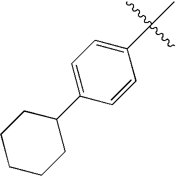 | 468.0 | δ 8.06 (d, J = 10.99 Hz, 1H), 7.16 (d, J = 8.55 Hz, 2H), 6.96 (d, J = 8.79 Hz, 2H), 6.92 (d, J = 8.30 Hz, 1H), 3.65 (s, 3H), 1.67 (m, b, 5H), 1.24 (m, b, 5H) | 0.0335 |
| 5 | 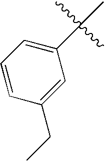 | 414.2 | δ 1.08 (t, 3 H) 2.53 (q, J = 7.56 Hz, 2H) 3.69 (s, 3 H) 6.80 (dd, J = 8.66, 3.05 Hz, 1 H) 6.98 (m, 3 H) 7.22 (t, 1H) 8.08 (d,J = 10.98 Hz, 1 H) 11.77 (s, 1H) 16.11 (s, 1H) | 0.0625 |
| 6 | 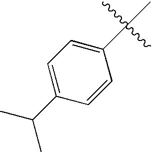 | 428.1 | δ 1.12 (d, J = 6.83 Hz, 6 H) 2.82 (m, 1H) 3.67 (s, 3 H) 6.97 (m, 3 H) 7.19 (d, J = 8.54 Hz, 2 H) 8.07 (d, J = 10.98 Hz, 1 H) 11.66 (s, 1 H) 16.10 (s, 1 H) | 0.0135 |

^{a 1}H-NMR (400 MHz, DMSO)

### Intermediate 1

### 3-(4-Fluoro-3-methoxy-phenyl)-acrylic acid

The title compound was prepared according to the following reaction: 4-fluoro-3-methoxy-benzaldehyde (50.0 g, 324 mmol) was refluxed with malonic acid (50.6 g, 487 mmol) in a mixture of piperidine (6.72 mL) and pyridine (224 mL) for 18 hours. The mix was concentrated to one half volume. 1N HCl was added until a solid precipitated and then H₂O (˜200 mL) was added. The solid was filtered. The filter cake was collected, slurried in H₂O (˜200 mL) at room temperature for fifteen minutes, and filtered again. The filter cake was rinsed with H₂O and then dried in a vacuum oven to give the title compound (58.27 g, 91.6% yield) as a chalky solid.

### Intermediate 2

### 3-Chloro-6-fluoro-5-methoxy-benzo[b]thiophene-2-carbonyl chloride

Intermediate 1 (58.3 g, 297 mmol) was dissolved in a mixture of pyridine (2.28 mL, 28.2 mmol), DMF (21.8 mL, 282 mmol), and chlorobenzene (371 mL) in an argon purged, round bottom, 2-necked flask fitted with a reflux condenser and an addition funnel. The mix was heated to reflux. Thionyl chloride (110 mL, 1515 mmol) was added dropwise to the mixture via the addition funnel over a period of 2.5 hours. The reaction was stirred at reflux for 18 hours. The reaction was allowed to cool to room temperature and then was concentrated en vacuo. The residue was dissolved in toluene and re-concentrated en vacuo to azeotrope residual pyridine. The residue was then dissolved in CH₂Cl₂ (˜300 mL) and then was diluted with an excess of hexanes (˜900 mL). The dilution was concentrated to about one half volume to give a precipitate. The solid precipitate was filtered, collected, and dried en vacuo to give the title compound (64.9 g, 78.3% yield) as an orange solid. ¹H-NMR (400 MHz, CDCl₃) δ 4.02 (s, 3H), 7.41 (d, J=7.81 Hz, 1H), 7.54 (d, J=10.00 Hz, 1H).

### Intermediate 3

### 3-Chloro-6-fluoro-5-methoxy-benzo[b]thiophene-2-carboxylic acid isopropyl ester

Intermediate 2 (64.9 g, 232 mmol) was stirred with triethylamine (64.8 mL, 465 mmol) and catalytic DMAP in isopropanol (290 mL) at 80 C for 18 hours. The reaction was allowed to cool to room temperature and then was concentrated en vacuo. The residue was dissolved in EtOAc and filtered though filter paper. The filtrate was washed with saturated aq. NaHCO₃ and then brine. The organics were dried over Na₂SO₄, filtered through celite, and concentrated en vacuo to give the title compound (50.37 g, 71.6% yield) as an orange-brown solid. ¹H-NMR (400 MHz, CDCl₃) δ 1.40 (d, J=6.34 Hz, 6H), 4.00 (s, 3H), 5.27 (d, 1H), 7.39 (d, J=7.81 Hz, 1H), 7.48 (d, J=10.00 Hz, 1H).

### Intermediate 4

### 3-Chloro-6-fluoro-5-methoxy-1-oxo-1H-1λ⁴-benzo[b]thiophene-2-carboxylic acid isopropyl ester

To a 0° C. solution of intermediate 3 (50.37 g, 166.4 mmol) in dichloromethane (100 mL) and TFA (100 mL) was added hydrogen peroxide (30% wt., 18.9 mL, 166 mmol). The cold bath was removed and the reaction was stirred at room temperature for three hours. The reaction was chilled to 0 C in an ice bath and then was slowly poured into a 0° C. solution of saturated aqueous sodium bisulfite. The quenched mixture was extracted three times with EtOAc. The organics were filtered, washed with brine, dried over Na₂SO₄, filtered through celite, and concentrated. The crude product was chromatographed over SiO₂ (0-10% Et₂O—CH₂Cl₂ gradient elution) to give the title compound (16.5 g, 31.1% yield) as an orange solid. MS: M+1=319.0 (APCI). ¹H-NMR (400 MHz, CDCl₃) δ 1.41 (m, 6H), 4.03 (s, 3H), 5.28 (m, 1H), 7.30 (d, J=7.08 Hz, 1H), 7.66 (d, J=8.78 Hz, 1H).

### Intermediate 5

### 3-(4-Chloro-phenoxy)-6-fluoro-5-methoxy-benzo[b]thiophene-2-carboxylic acid isopropyl ester

To a stirring solution of 4-chlorophenol (0.133 g, 1.035 mmol) in anhydrous THF (2.0 mL) at room temperature was added sodium hydride (0.0294 g, 1.22 mmol). The reaction fizzed and turned chalky gray. The mix was stirred at room temperature for five minutes and then was added to a stirring solution of Intermediate 4 (0.300 g, 0.941 mmol) in anhydrous THF (2.7 mL). The reaction was stirred at room temperature for ten minutes. Sodium iodide (0.353 g, 2.35 mmol) and then TMSCl (0.299 mL, 2.35 mmol) were added. The reaction turned dark red-brown. The mix was stirred at room temperature for five minutes. The reaction was quenched with saturated aq. sodium thiosulfate until all of the red-brown color dissipated to a light yellow. The quenched mix was diluted with H₂O and extracted three times with EtOAc. The organics were washed with brine, dried over Na₂SO₄, filtered through celite, and concentrated. The product was purified by silica gel flash chromatoghraphy (0-10% EtOAc-hexanes) to give the title compound (0.262 g, 70.5% yield) as a waxy, colorless solid. MS: M+1=395.0 (APCI).). ¹H-NMR (400 MHz, CDCl₃) δ 1.16 (d, J=6.34 Hz, 6H), 3.85 (s, 3H), 5.09 (m, 1H), 6.86 (d, J=9.03 Hz, 2H), 7.07 (d, J=7.81 Hz, 1H), 7.23 (m, 2H), 7.50 (d, J=10.25 Hz, 1H).

### Intermediate 6

### 3-(4-Chloro-phenoxy)-6-fluoro-5-methoxy-benzo[b]thiophene-2-carboxylic acid

A mixture of Intermediate 5 (0.240 g, 0.608 mmol) and 1N LiOH (1.6 mL) were stirred at 80° C. in dioxane (2.4 mL) for two hours. The mix was allowed to cool to room temperature and then was acidified to pH˜1 with 1N HCl. The acidified mix was diluted with H₂O to give a solid precipitate. The solid was filtered, rinsed with H₂O, and dried overnight in a vacuum oven to give the title compound (0.116 g, 54.1% yield) as a fluffly white solid. MS: M+1=353.0 (APCI). NMR (400 MHz, DMSO) δ ppm 3.77 (s, 3H) 6.93 (m, 2H), 7.12 (d, J=8.05 Hz, 1H), 7.34 (m, 2H), 8.03 (d, J=10.98 Hz, 1H), 13.39 (s, 1H).

### Example 1

### 3-(4-Chloro-phenoxy)-6-fluoro-5-methoxy-benzo[b]thiophene-2-carboxylic acid (2H-tetrazol-5-yl)-amide

Intermediate 6 (0.113 g, 0.320 mmol) was mixed with—but did not dissolve in anhydrous dichloromethane (1.6 mL). About three drops of DMF were added. Oxalyl chloride (0.0307 mL, 0.352 mmol) was added via syringe. The mix fizzed and slowly became homogeneous. The reaction was stirred at room temperature for ten minutes. 5-Amino tetrazole (0.0681 g, 0.801 mmol), triethylamine (0.112 mL, 0.801 mmol), and finally acetonitrile (1.6 mL) were added. The reaction was stirred at 80° C. for 30 minutes. The reaction vessel was opened and allowed to boil nearly dry. The heating bath was removed and the mix was allowed to cool to room temperature. The remaining tar was taken up in a minimum of acetonitrile (˜0.4 mL). The mix was diluted with H₂O (˜3 mL) and acidified with 1N HCl until a fluffy white solid precipitated. The solid was filtered and rinsed with H2O. The cake was collected and slurried in a minimum of MeOH for 30 minutes. The slurry was filtered and the cake was rinsed with a minimum of MeOH. The solid was dried overnight in a vacuum oven to give the title compound (0.134 g, 64.0% yield) as a white solid. MS: M+1=420.0 (APCI). ¹H-NMR (400 MHz, DMSO) δ 3.74 (s, 3H) 7.03 (d, J=9.27 Hz, 2H) 7.07 (d, J=8.05 Hz, 1H) 7.37 (d, J=9.03 Hz, 2H) 8.11 (d, J=10.98 Hz, 1H).

The title compounds of Examples 2-6 were synthesized in a manner analogous to Example 1 by replacing 4-chloro-phenol with an appropriately substituted phenol (e.g., 4-cyclopentyloxy-phenol).

### Example 2

### 3-(4-Cyclopentyloxy-phenoxy)-6-fluoro-5-methoxy-benzo[b]thiophene-2-carboxylic acid (2H-tetrazol-5-yl)-amide

### Example 3

### 6-Fluoro-5-methoxy-3-phenoxy-benzo[b]thiophene-2-carboxylic acid iminomethyl-amide

### Example 4

### 3-(4-Cyclohexyl-phenoxy)-6-fluoro-5-methoxy-benzo[b]thiophene-2-carboxylic acid (2H-tetrazol-5-yl)-amide

### Example 5

### 3-(3-Ethyl-phenoxy)-6-fluoro-5-methoxy-benzo[b]thiophene-2-carboxylic acid (2H-tetrazol-5-yl)-amide

### Example 6

### 6-Fluoro-3-(4-isopropyl-phenoxy)-5-methoxy-benzo[b]thiophene-2-carboxylic acid (2H-tetrazol-5-yl)-amide

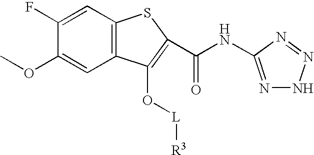

| | | | | IC₅₀ |
|---|---|---|---|---|
| Ex. | —L—R³ | MS (M + 1) | NMR^{a} | (μM) |
| 7 | 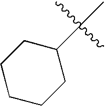 | 392.1 | δ 1.26 (m, 3H), 1.47 (m, 1H), 1.61 (m, 2H), 1.71 (m, 2H), 2.02 (m, 2H), 3.95 (s, 3H), 4.53 (m, 1H), 7.43 (d, J = 8.05 Hz, 1H), 8.00 (d, J = 10.98 Hz, 1H), 11.23 (s, 1H), 16.21 (s, 1H). | 0.0105 |
| 8 | 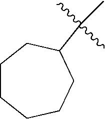 | 406.1 | δ 1.36 (m, 2 H) 1.50 (s, 4 H) 1.67 (m, 2 H) 1.84 (m, 2 H) 2.08 (m, 2 H) 3.95 (s, 3 H) 4.75 (m, 1 H) 7.39 (d, J = 8.05 Hz, 1 H) 8.00 (d, J = 11.22 Hz, 1 H) 11.19 (s, 1 H) 16.16 (s, 1 H) | 0.0034 |
| 9 | 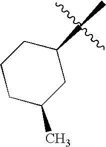 | 406.1 | δ 0.78 (m, 1 H), 0.86 (d, J = 6.59 Hz, 3H), 1.24 (m, 2 H), 1.48 (m, 3 H), 1.71 (d, J = 13.18 Hz, 1 H), 2.10 (d, J = 12.20 Hz, 2 H), 3.95 (s, 3 H), 4.50 (m, 1 H), 7.43 (d, J = 8.05 Hz, 1 H), 8.01 (d, J = 10.98 Hz, 1 H), 11.18 (s, 1 H), 16.23 (s, 1 H) | 0.0038 |
| 10 | 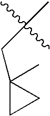 | 378.1 | δ 0.46 (m, 2 H), 0.61 (m, 2 H), 1.34 (s, 3 H), 3.96 (s, 3 H) 4.25 (s, 2 H), 7.44 (d, J = 8.05 Hz, 1 H), 8.01 (d, J = 10.98 Hz, 1 H), 11.11 (s, 1 H), 16.17 (s, 1 H) | 0.0140 |
| 11 | 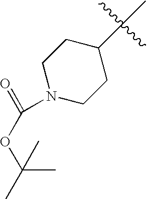 | 491.1 | δ 1.35 (s, 9H), 1.68 (m, 2H), 1.98 (m, 2H), 2.95 (m, 2H), 3.80 (m, 2H), 3.96 (s, 3H), 4.67 (m, 1H), 7.44 (d, J = 8.05 Hz, 1H), 8.01 (d, J = 10.98 Hz, 1H) | 0.0160 |
| 12 | 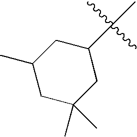 | 434.1 | δ 0.79 (s, 3H), 0.84 (d, J = 6.59 Hz, 3H), 0.91 (s, 3H), 1.12 (q, J = 11.87 Hz, 1H), 1.27 (d, J = 13.18 Hz, 1H), 1.39 (t, J = 11.83 Hz, 1H), 1.60 (m, 1H), 1.92 (m, 1H), 2.05 (m, 1H), 3.94 (s, 3H), 4.69 (s, 1H), 7.42 (d, J = 8.05 Hz, 1H), 8.00 (d, J = 10.98 Hz, 1H), 11.20 (s, 1 H), 16.19 (s, 1 H) | 0.0053 |
| 13 | 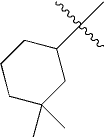 | 420.1 | δ 0.80 (s, 3H), 0.92 (s, 3H), 1.11 (m, 1H), 1.25 (m, 1H), 1.46 (m, 3H), 1.60 (m, 1H), 1.92 (d, J = 11.96 Hz, 1H), 2.05 (m, 1H), 3.94 (s, 3H), 4.64 (m 1H), 7.41 (d, J = 8.05 Hz, 1H), 8.01 (d, J = 10.98 Hz, 1H), 11.20 (s, 1H), 16.20 (m, 1H) | 0.0065 |
| 14 | 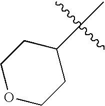 | 394.1 | δ 1.79 (m, 2H), 2.00 (d, J = 16.59 Hz, 2H), 3.36 (m, 2H), 3.87 (m, 2H), 3.96 (s, 3H), 4.71 (m, 1H), 7.46 (d, J = 8.05 Hz, 1H), 8.01 (d, J = 10.98 Hz, 1H), 11.37 (m, 1H), 16.23 (m, 1H) | 0.0625 |
| 15 | 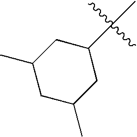 | 420.1 | | 0.0022 |
| 16 | 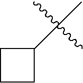 | 364.0 | δ 1.49 (m, 1H), 1.72 (m, 1H), 2.31 (m, 4H), 3.95 (s, 3H), 5.03 (m, 1H), 7.42 (d, J = 8.05 Hz, 1H), 8.00 (d, J = 10.98 Hz, 1H), 11.24 (s, 1H), 16.22 (s, 1H) | 0.0325 |

^{a1}H-NMR (400 MHz, DMSO)

### Intermediate 7

### 3-Cyclohexyloxy-6-fluoro-5-methoxy-benzo[b]thiophene-2-carboxylic acid isopropyl ester

To a −78° C. solution of cyclohexanol (1.82 mL, 17.3 mmol) in anhydrous THF (˜20 mL) was added nBuLi (1.6 M in hexanes, 10.3 mL, 16.5 mmol) dropwise. The cold bath was removed and the mixture was allowed to warm to room temperature. In a separate reaction flask, compound intermediate 4 (5.00 g, 15.7 mmol) was mixed with—but did not dissolve in anhydrous THF (˜50 mL). The alkoxide solution was added to the intermediate 4 mixture dropwise via syringe. The reaction turned yellow and became homogeneous. The reaction was stirred at room temperature for fifteen minutes. Sodium iodide (5.90 g, 39.4 mmol) and then trimethylsilyl chloride (4.98 mL, 39.2 mmol) were added. The reaction turned red-brown. The mix was stirred at room temperature for twenty-five minutes. The reaction was quenched with saturated aq. sodium thiosulfate until the red-brown color turned light yellow. The quench was diluted with sat. aq. NaHCO₃ and extracted three times with EtOAc. The organics were washed with brine, dried over Na₂SO₄, filtered through celite, and concentrated. The crude product was purified by silica gel flash chromatography (0-10% Et₂O-hexanes, Isco CombiFlash™ gradient elution (Isco, Inc., Lincoln, Nebr.), TLC in 10% EtOAc-hexanes) to give the title compound (3.33 g, 57.9% yield) as a light green oil. MS: M+1=367.1 (APCI). ¹H-NMR (400 MHz, CDCl₃) ppm 1.30 (m, 3H), 1.37 (d, J=6.10 Hz, 6H), 1.59 (m, 3H) 1.81 (m, 2H), 2.05 (m, 2H), 3.96 (s, 3H), 4.49 (m, 1H), 5.24 (m, 1H), 7.31 (d, J=8.05 Hz, 1H), 7.38 (d, J=10.25 Hz, 1H).

### Intermediate 8

### 3-Cyclohexyloxy-6-fluoro-5-methoxy-benzo[b]thiophene-2-carboxylic acid

A mixture of Intermediate 7 (3.33 g, 9.09 mmol) and 1N LiOH (18 mL) were stirred at 80° C. in dioxane (27 mL) for two hours. The mix was allowed to cool to room temperature and then was acidified to pH˜1 with 1N HCl. The acidified mix was diluted with H₂O to give a solid precipitate. The solid was filtered, rinsed with H₂O, and dried overnight in a vacuum oven to give the title compound (2.80 g, 95.2% yield) as a fluffly white solid. MS: M+1=325.0 (APCI). ¹H-NMR (400 MHz, DMSO) ppm 1.24 (m, 3H), 1.55 (m, 3H), 1.72 (m, 2H), 1.89 (m, 2H), 3.90 (s, 3H), 4.47 (m, 1H), 7.34 (d, J=8.05 Hz, 1H), 7.87 (d, J=10.98 Hz, 1H), 13.14 (s, 1H).

### Example 7

### 3-Cyclohexyloxy-6-fluoro-5-methoxy-benzo[b]thiophene-2-carboxylic acid (2H-tetrazol-5-yl)-amide

Intermediate 8 (2.70 g, 8.32 mmol) was dissolved in anhydrous CH₂Cl₂ (25 mL) in an argon-purged flask. A catalytic drop of DMF followed by oxalyl chloride (0.799 mL, 9.16 mmol) were added via syringe. The reaction was stirred at room temperature for 30 minutes. Acetonitrile (25 mL) and then 5-aminotetrazole (1.77 g, 20.8 mmol) and triethylamine (2.90 mL, 20.8 mmol) were added. The reaction was stirred at reflux for 30 minutes and then was allowed to cool to room temperature. The reaction was diluted with H₂O and acidified with 1N HCl until a solid precipitated. The solid was filtered and rinsed with H₂O. The filter cake was slurried in a minimum MeOH, filtered again, and dried en vacuo to give the title product (3.04 g, 93.2% yield).

The title compounds of Examples 8-16 were synthesized in a manner analogous to Example 7 by replacing cyclohexanol with an appropriate alcohol.

### Example 8

### 3-Cycloheptyloxy-6-fluoro-5-methoxy-benzo[b]thiophene-2-carboxylic acid (2H-tetrazol-5-yl)-amide

### Example 9

### cis-(±)-6-Fluoro-5-methoxy-3-(3-methyl-cyclohexyloxy)-benzo[b]thiophene-2-carboxylic acid (2H-tetrazol-5-yl)-amide

### Example 10

### 6-Fluoro-5-methoxy-3-(1-methyl-cyclopropylmethoxy)-benzo[b]thiophene-2-carboxylic acid (21H-tetrazol-5-yl)-amide

### Example 11

### 4-[6-Fluoro-5-methoxy-2-(2H-tetrazol-5-ylcarbamoyl)-benzo[b]thiophen-3-yloxy]-piperidine-1-carboxylic acid tert-butyl ester

### Example 12

### 6-Fluoro-5-methoxy-3-(3,3,5-trimethyl-cyclohexyloxy)-benzo[b]thiophene-2-carboxylic acid (2H-tetrazol-5-yl)-amide

### Example 13

### 3-(3,3-Dimethyl-cyclohexyloxy)-6-fluoro-5-methoxy-benzo[b]thiophene-2-carboxylic acid (2H-tetrazol-5-yl)-amide

### Example 14

### 6-Fluoro-5-methoxy-3-(tetrahydro-pyran-4-yloxy)-benzo[b]thiophene-2-carboxylic acid (2H-tetrazol-5-yl)-amide

### Example 15

### 3-(3,5-Dimethyl-cyclohexyloxy)-6-fluoro-5-methoxy-benzo[b]thiophene-2-carboxylic acid (2H-tetrazol-5-yl)-amide

### Example 16

### 3-Cyclobutoxy-6-fluoro-5-methoxy-benzo[b]thiophene-2-carboxylic acid (2H-tetrazol-5-yl)-amide

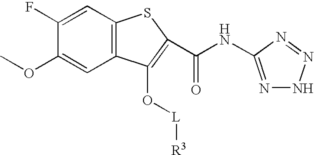

| | | MS | | IC₅₀ |
|---|---|---|---|---|
| Ex. | —L—R³ | (M + 1) | NMR^{a} | (μM) |
| 17 | 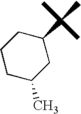 | 406.2 | δ 0.80 (d, J = 6.59 Hz, 3H), 1.05 (m, 1H), 1.34 (m, 1H), 1.55 (m, 4H), 1.93 (m, 3H), 3.93 (s, 3H), 4.83 (m, 1H), 7.40 (d, J = 8.05 Hz, 1H), 8.00 (d, J = 10.98 Hz, 1H), 11.50 (s, 1H), 16.16 (s, 1H) | 0.0350 |

^{a 1}H-NMR (400 MHz, DMSO)

### Intermediate 9

### 6-Fluoro-3-hydroxy-5-methoxy-1-oxo-1H-1λ⁴-benzo[b]thiophene-2-carboxylic acid isopropyl ester.

Intermediate 4 (1.50 g, 4.71 mmol) was mixed with—but did not dissolve in dioxane (15 mL). H₂O (5 mL) and then 1N LiOH (4.7 mL) were added. The reaction turned brown and became more homogeneous. The mix was stirred at room temperature for twenty minutes. 1N LiOH (5 mL) was added and the reaction was stirred at room temperature an additional 30 minutes. The reaction was acidified to pH=1 with 1N HCl and then was diluted with H₂O to give a yellow solid precipitate. The solid was filtered and rinsed with H₂O. The cake was collected and dried in the vacuum oven to give 922 mg of the title compound. HPLC analysis indicated desired product was present in the aqueous filtrate. The filtrate was extracted twice with CH₂Cl₂. The extracts were concentrated and dried under vacuum to give another 422 mg of desired product. MS: M+1=301.1 (APCI).

### Intermediate 10

### 6-Fluoro-3-hydroxy-5-methoxy-benzo[b]thiophene-2-carboxylic acid isopropyl ester

Intermediate 9 (0.915 g, 3.05 mmol) was mixed with—but did not dissolve in anhydrous acetonitrile (15 mL) in a round bottom flask stirring at room temperature. Sodium iodide (0.685 g, 4.57 mmol) and then TMSCl (0.580 mL, 4.57 mmol) were added. The reaction turned dark brown red. The mix was stirred at room temperature for ten minutes. The reaction was quenched dropwise with saturated aq. sodium thiosulfate until the red-brown color dissipated to yellow. The mix was then diluted with H₂O to give a white precipitate. The solid was filtered, rinsed with H₂O, and dried under vacuum to give the title compound (0.660 g, 76.2% yield) as a very light green, fluffy, electrostatic solid. MS: M−1=283.0 (APCI). ¹H-NMR (400 MHz, CDCL₃) ppm 1.39 (d, J=6.34 Hz, 6H), 3.96 (s, 3H), 5.28 (m, 1H), 7.39 (m, 2H), 10.23 (s, 1H).

### Intermediate 11

### 6-Fluoro-5-methoxy-3-(3-methyl-cyclohexyloxy)-benzo[b]thiophene-2-carboxylic acid isopropyl ester

Intermediate 10 (0.880 g, 3.10 mmol) was combined with cis-3-methylcyclohexanol (0.424 g, 3.71 mmol), Ps-PPh₃ (Polystyrene-triphenylphosphine) (4.22 g), and DEAD (0.809 g, 4.64 mmol) in anhydrous THF (25 mL). The reaction was stirred at room temperature for 18 hours. The mix was filtered and the resin cake was rinsed with dichloromethane. The filtrate was concentrated to give the title compound (0.618 g, 52.5% yield) as a colorless oil. MS: M+1=381.2 (APCI).

### Intermediate 12

### 6-Fluoro-5-methoxy-3-(3-methyl-cyclohexyloxy)-benzo[b]thiophene-2-carboxylic acid

A mixture of Intermediate 11 (0.615 g, 1.62 mmol) and 1N LiOH (4 mL) were stirred at 80° C. in dioxane (6 mL) for two hours. The mix was allowed to cool to room temperature and then was acidified to pH˜1 with 1N HCl. The acidified mix was diluted with H₂O to give a solid precipitate. The solid was filtered, rinsed with H₂O, and dried overnight in a vacuum oven to give the title compound (0.533 g, 97.4% yield) as a fluffly white solid. MS: M+1=339.1 (APCI). ¹H-NMR (400 MHz, CDCl₃) ppm 0.95 (d, J=6.59 Hz, 3H), 1.10 (m, 1H), 1.39 (m, 1H), 1.63 (m, 2H), 1.80 (m, 2H), 2.06 (m, 3H), 3.95 (s, 3H), 4.94 (m, 1H), 7.32 (d, J=7.81 Hz, 1H), 7.45 (d, J=10.25 Hz, 1H).

### Example 17

### trans-(±)-6-Fluoro-5-methoxy-3-(3-methyl-cyclohexyloxy)-benzo[b]thiophene-2-carboxylic acid (2H-tetrazol-5-yl)-amide

Intermediate 12 (0.528 g, 1.56 mmol) was mixed with—but did not dissolve in anhydrous dichloromethane (8 mL). About three drops of DMF were added. Oxalyl chloride (0.150 mL, 0.1.72 mmol) was added via syringe. The mix fizzed and slowly became homogeneous. The reaction was stirred at room temperature for ten minutes. 5-Amino tetrazole (0.332 g, 3.90 mmol), triethylamine (0.543 mL, 3.90 mmol), and finally acetonitrile (8 mL) were added. The reaction was stirred at 80° C. for 30 minutes. The reaction vessel was opened and allowed to boil nearly dry. The heating bath was removed and the mix was allowed to cool to room temperature. The remaining tar was taken up in a minimum of acetonitrile (˜1 mL). The mix was diluted with H₂O (˜15 mL) and acidified with 1N HCl until a fluffy white solid precipitated. The solid was filtered and rinsed with H₂O. The cake was collected and slurried in a minimum of MeOH for 30 minutes. The slurry was filtered and the cake was rinsed with a minimum of MeOH. The solid was dried overnight in a vacuum oven to give the title compound (0.280 g, 44.3% yield) as a white solid. MS: M+1=406.2 (APCI). ¹H-NMR (400 MHz, DMSO) δ 0.80 (d, J=6.59 Hz, 3H), 1.05 (m, 1H), 1.34 (m, 1H), 1.55 (m, 4H), 1.93 (m, 3H), 3.93 (s, 3H), 4.83 (m, 1H), 7.40 (d, J=8.05 Hz, 1H), 8.00 (d, J=10.98 Hz, 1H), 11.50 (s, 1H), 16.16 (s, 1H).

### Intermediate 13

### 3-(3-methoxy-4-flouro-phenyl) acrylic acid

A solution of 3-methoxy-4-fluoro benzaldehyde (2.4 g, 15 mmol), malonic acid (2.34 g, 22 mmol), piperidine (0.9 mL), and pyridine (30 mL) was heated to 80° C. for a period of one hour, then heated to reflux for an additional 3 hours. The solution was cooled to ambient temperature and approximately half of the solvent was removed under reduced pressure. The solution was poured on to 20 ml 1 N HCl and the resulting precipitate removed by filtration. The solid was dissolved in ethyl acetate and extracted with HCl (1N), water, and brine. The solution was dried over MgSO₄ and concentrated under reduced pressure. Recrystallization in ethyl acetate afforded the title compound (1.4 g, 47%). ¹H-NMR—(400 MHz, D₆ DMSO) δ,12.3 (s,1H), 7.53 (m, 2H), 7.25 (m, 2H) 6.57(d, j=15.93 Hz, 1H), 3.86(s, 3H).

### Intermediate 14

### 3-Chloro-6-fluoro-5-methoxy-benzo[b]thiophene-2-carboxylic acid methyl ester

A slurry of Intermediate 13 (1.4 g, 7 mmol), pyridine (53 μL, 0.65 mmol), dimethylformamide (500 μL, 6.6 mmol), thionyl chloride (2.65 mL and chlorobenzene (15 mL) was heated to reflux for a period of 21 hours. The reaction was cooled to room temperature and the solvent removed under reduced pressure. The residue was dissolved in dichloromethane (15 mL) and treated with methanol (15 mL). The resulting solid was removed by filtration and washed with methanol to afford the title compound (0.92 g, 47%). ¹H-NMR—(400 MHz, D₆ DMSO) δ, 8.12(d, J=10.8 Hz, 1H), 7.512(d, J=8.06 Hz, 1H), 3.96(s,3H), 3.87(s, 3H).

### Intermediate 15

### 3-Chloro-6-fluoro-5-methoxy-benzo[b]thiophene-2-carboxylic acid (1H-tetrazol-5-yl)-amide

To a solution of Intermediate 14 (5.40 g, 19.7 mmol) in THF (40 mL) was added 1N NaOH (40 mL) and H₂O (300 mL). The mix was stirred and heated to 65-75° C. for two hours and then allowed to cool to room temperature. The reaction mixture was washed twice with EtOAc and then was acidified to give an off-white solid precipitate. The precipitate was extracted several times with EtOAc. The organics were washed with brine and concentrated to give 3-chloro-6-fluoro-5-methoxy-benzo[b]thiophene-2-carboxylic acid (3.10 g, 60% yield). To a room temperature solution of 3-chloro-6-fluoro-5-methoxy-benzo[b]thiophene-2-carboxylic acid (2.02 g, 7.75 mmol) in CH₂Cl₂ was added oxalyl chloride (1.35 mL, 15.5 mmol) followed by 2-3 drops of DMF. The reaction was stirred at room temperature for two hours and then was concentrated. The residue was dissolved in CH₃CN (60 mL). Triethylamine (1.08 mL, 7.75 mmol) followed by 5-aminotetrazole (0.725 g, 9.52 mmol) were added. The reaction was stirred at 60° C for 16 hours and then was cooled to room temperature. The mix was diluted with H₂O (˜30 mL) to give a solid precipitate. The solid was filtered, washed with H₂O and CH₃CN to give the title product (2.35 g, 92%). MS: M+1=328 (APCI).

### Intermediate 16

### 3-(2-Flouro 3-methoxy-phenyl) acrylic acid

A solution of 2 fluoro-anisole (6.0 g, 47 mmol) and N,N,N′,N′-teteramethylethylenediamine in THF (250 mL) was cooled to −78° C. and treated dropwise with sec-butyl lithium (1.3 M/cyclohexane, 37.9 mL, 49 mmol). The mixture was stirred for 2 hours at −78° C. and then treated with dimethylformamide (3.6 mL, 47 mmol). Stirring continued for an additional 10 minutes and then acetic acid (20 mL) followed by water (150 mL) was added to the reaction. The reaction was warmed to ambient temperature and then diluted with ethyl acetate. The organic layer was extracted first with 1N HCl, and then with brine. The solution was dried over MgSO₄ and concentrated under reduced pressure to afford crude 2 flouro-3-methoxybenzaldehyde (4.7 g, 64%). In a manner analogous to that described in Intermediate 13, 2 flouro-3-methoxybenzaldehyde (4.7 g, 30 mmol), malonic acid (4.76 g, 45 mmol), pyridine (50 mL) and piperidine (1.5 mL) afforded the title product (2.5 g, 42%). ¹H-NMR—(400 MHz, D₆ DMSO) δ,7.65(d,J=16.1 Hz,1H), 7.34 (m,1H), 7.18(m,2H), 6.58(d, J=16.1 Hz, 1H), 3.83(s,3H).

### Intermediate 17

### 3-Chloro-4-fluoro-5-methoxy-benzo[b]thiophene-2-carboxylic acid methyl ester

In a manner analogous to that described in Intermediate 14, Intermediate 16 (2.5 g, 12 mmol), pyridine (0.103 mL, 1.2 mmol), dimethylformamide (0.83 mL, 10 mmol), thionyl chloride (4.1 mL) and chlorobenzene (30 mL) afforded the title compound (0.72 g, 18%). ¹H-NMR—(400 MHz, D₆ DMSO) δ,7.90(d,J=1.5 Hz,1H), 7.87(m,1H), 3.91(s, 3H), 3.87(s,1H).

### Intermediate 18

### 3-Chloro-4-fluoro-5-methoxy-benzo[b]thiophene-2-carboxylic acid; (1H-tetrazol-5-yl)-amide

In a manner similar to that described in Intermediate 15, Intermediate 17 was converted to the title product (1.8 g, 56%). MS: M+1=328.

### Intermediate 19

### 3-Chloro-7-fluoro-6-methoxy-benzo[b]thiophene-2-carbonyl chloride

A mixture of 3-(3-fluoro-4-methoxy-phenyl)-acrylic acid (Baddar et al. (1976) J. Indian Chem. Soc. 53: 1053-1058) (5.0 g, 25 mmol), pyridine (0.21 mL, 2.5 mmol), dimethylformamide (1.74 mL, 22 mmol), thionyl chloride (9.3 mL and chlorobenzene (100 mL) was heated to reflux for 21 hours. The reaction was cooled to room temperature and the solvent removed under reduced pressure. The residue was redissolved in toluene and concentrated under reduced pressure. The title compound was recrystallized from toluene/hexanes to afford the title compound (4.7 g, 66%). ¹H-NMR—(Methyl ester) (400 MHz, D₆ DMSO) δ 7.91 (d, J=7.57 Hz, 1H), 7.72 (d, J=11.2 Hz, 1H), 3.91 (s, 3H), 3.84 (s, 3H).

### Intermediate 20

### 3-Chloro-7-fluoro-6-methoxy-benzo[b]thiophene-2-carboxylic acid (1H-tetrazol-5-yl)-amide

A solution of Intermediate 19 (3.7 g, 13.3 mmol), amino teterazole (1.25 g, 14.7 mmol), and triethyl amine (2.4 mL, 17.4 mmol) in acetonitrile (60 mL) was heated to reflux for a period 18 hours. The reaction was cooled and the solvent removed under reduced pressure. Water was added to the residue and the resulting solid collected by filtration. The dried solid was recrystallized from dichloromethane/methanol to afford the title compound (1.38 g, 31%) ¹H-NMR—(400 MHz, D₆ DMSO) δ 7.98 (d, J=7.5 Hz, 1H), 7.75(d, J=11.2 Hz, 1H), 3.92 (s,3H). MS M⁺¹=327.

### Synthesis of Examples 18-40

The solutions of the desired thiols (R¹-L-SH) were prepared such that the final molarity of the solution (DMF) was 0.66 M of the desired thiols (1 equivalent) and 0.66 M DBU. To the appropriate vial (2 dram), Intermediate 15, 17, or 20 (1.5 ml, 0.11 mmol) and the desired thiol solution (0.5 mL, 0.33 mmol). The vials were capped and heated to 80° C. with shaking for 10 hours. The reactions were cooled and transferred to individual vessels of a Bohdan Mini Block. The vials were washed with DMF (0.5 ml) and the wash was transferred to the appropriate vessel in the Mini Block. ArgoPore®-Isocyanate capture resin (0.20 g) (Argonaut Technologies, Inc., Foster City, Calif.) was added to each vessel and mixed for 2 hours. The resulting solutions were filtered to remove the resin. The resin was then washed with DMF (0.5 mL). The reactions were then treated with acetic acid (0.2M methanol, 0.5 ml) and then the solvent was removed under reduced pressure. The residue was dissolved in methanol (2 mL) and transferred into fresh vessels on the Bohdan Mini Block. Argonaut MP-TsOH capture resin (0.20 g) (Argonaut Technologies, Inc., Foster City, Calif.) was added to each vessel and mixed for 18 hours. The resin was removed by filtration, the resin was washed with methanol (0.4 mL) and DMF (1.5 mL). The solvent was removed under reduced pressure and the title compounds purified by reverse phase chromatography.

For the syntheses of Examples 25 and 34-36, the Argonaut MP-TsOH capture resin was removed by flitration, and washed with methanol (0.4 ml) and DMF (1.5 ml) as described above. The solvent was removed under reduced pressure and the residue was redissolved in THF (1.0 mL). The solution was then treated with sodium hydroxide (1 mL, 1.0N) and allow to shake for 18 hours. The solvent was removed under reduced pressure and the residue redissolved in water (0.5 mL) and treated with HCl (1 ml, 1.0N). The solution was extracted with ethyl acetate and the organic extracts were concentrated under reduced pressure. The title compounds were purified by reverse phase chromatography.

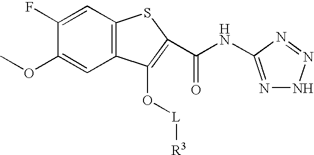

| | | MS | NMR^{a} | IC₅₀ |
|---|---|---|---|---|
| Ex. | —L—R³ | (M + 1) | % HPLC purity | (μM) |
| 18 | 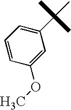 | 432 | HPLC 85% | 0.47 |
| 19 | 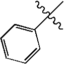 | 402.1 | δ 3.71 (s, 3 H) 7.22 (m, 6 H) 8.12 (d, J = 10.98 Hz, 1H) 12.60 (s, 1 H) 16.21 (s, 1 H) | 0.0325 |
| 20 | 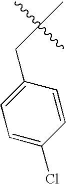 | 450.0 | δ 3.90 (s, 3H), 4.12 (s, 2H), 6.94 (d, J = 8.54 Hz, 2H), 7.08 (d, J = 8.54 Hz, 2H), 7.37 (d, J = 8.30 Hz, 1H), 8.06 (m, J = 10.74 Hz, 1H), 11.99 (s, 1H), 16.12 (s, 1H) | 0.0455 |

^{a 1}H-NMR (400 MHz, DMSO).
*HPLC Methodology: Column Devosil ODS-A C 18 , UV Detection: 254 nM
Solvent A Acetonitrile / 3% isopropyl alcohol
Solvent B H₂O / 3% isopropyl alcohol
Gradient 10 % A / 90% B to 100% A / 0% B over 10 minutes.

### Example 18

### 6-Fluoro-5-methoxy-3-(3-methoxy-phenylsulfanyl)-benzo[b]thiophene-2-carboxylic acid (1H-tetrazol-5-yl)-amide

### Example 19

### 6-Fluoro-5-methoxy-3-phenylsulfanyl-benzo[b]thiophene-2-carboxylic acid (1H-tetrazol-5-yl)-amide

### Example 20

### 3-(4-Chloro-phenylsulfanyl)-6-fluoro-5-methoxy-benzo[b]thiophene-2-carboxylic acid (1H-tetrazol-5-yl)-amide

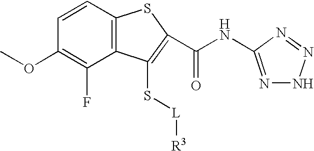

| | | MS | NMR^{a} or | IC₅₀ |
|---|---|---|---|---|
| Ex. | —L—R³ | (M − 1) | % HPLC Purity* | (μM) |
| 21 | 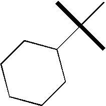 | 406 | 98 | 0.58 |
| 22 | 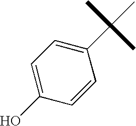 | 417 | 98 | 2.76 |
| 23 | 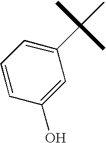 | 417 | 98 | 3.64 |
| 24 | 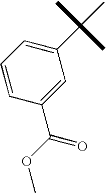 | 459 | 95 | 4.20 |
| 25 | 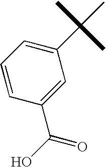 | 445 | 98 | 2.39 |
| 26 | 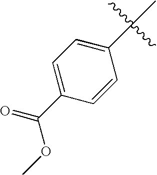 | 458 | 93 | 20.00 |
| 27 | 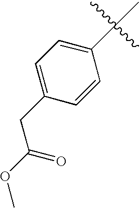 | 472 | 95 | 14.20 |
| 28 | 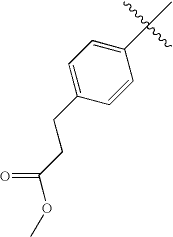 | 486 | 90 | 14.9 |
| 29 | 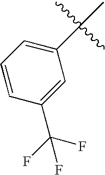 | 486 | 90 | 10.95 |
| 30 | 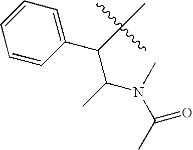 | 468 | 94 | 20.0 |
| 31 | 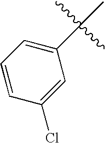 | 434 | 95 | 5.87 |
| 32 | 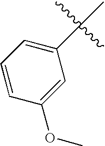 | 430 | 85 | 5.94 |
| 33 | 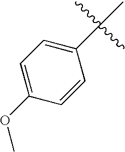 | 430 | 70 | 19.35 |
| 34 | 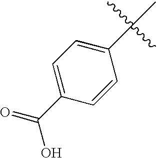 | 444 | 95 | 5.95 |
| 35 | 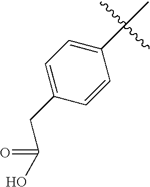 | 459 | 70 | 19.35 |
| 36 | 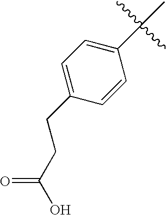 | 472 | 95 | 7.18 |
| 37 | 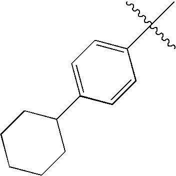 | 466.1 | δ 1.36 (m, 5H), 1.75 (m, 5H), 2.49 (m, 1H), 3.93 (s, 3H), 6.90 (d, J = 8.55 Hz, 2H), 7.22 (d, J = 8.55 Hz, 2H), 7.63 (m, 1 H) 8.00 (d, J = 8.79 Hz, 1H), 12.05 (s, 1H), 16.23 (s, 1 H). | 0.505 |
| 38 | 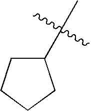 | 392 | 95 | 0.19 |

^{a 1}H-NMR (400 MHz, DMSO).
*HPLC Methodology: Column Devosil ODS-A C 18, UV Detection: 254 nM
Solvent A Acetonitrile / 3% isopropyl alcohol
Solvent B H₂O / 3% isopropyl alcohol
Gradient 10 % A /90% B to 100% A / 0% B over 10 min

### Example 21

### 3-Cyclohexylsulfanyl-4-fluoro-5-methoxy-benzo[b]thiophene-2-carboxylic acid (1H-tetrazol-5-yl)-amide

### Example 22

### 4-Fluoro-3-(4-hydroxy-phenylsulfanyl)-5-methoxy-benzo[b]thiophene-2-carboxylic acid (1H-tetrazol-5-yl)-amide

### Example 23

### 4-Fluoro-3-(3-hydroxy-phenylsulfanyl)-5-methoxy-benzo[b]thiophene-2-carboxylic acid (1H-tetrazol-5-yl)-amide

### Example 24

### 3-[4-Fluoro-5-methoxy-2-(1H-tetrazol-5-ylcarbamoyl)-benzo[b]thiophen-3-ylsulfanyl]-benzoic acid methyl ester

### Example 25

### 3-[4-Fluoro-5-methoxy-2-(1H-tetrazol-5-ylcarbamoyl)-benzo[b]thiophen-3-ylsulfanyl]-benzoic acid

### Example 26

### 4-[4-Fluoro-5-methoxy-2-(1H-tetrazol-5-ylcarbamoyl)-benzo[b]thiophen-3-ylsulfanyl]-benzoic acid methyl ester

### Example 27

### {4-[4-Fluoro-5-methoxy-2-(1H-tetrazol-5-ylcarbamoyl)-benzo[b]thiophen-3-ylsulfanyl]-phenyl}-acetic acid methyl ester

### Example 28

### 3-{4-[4-Fluoro-5-methoxy-2-(1H-tetrazol-5-ylcarbamoyl)-benzo[b]thiophen-3-ylsulfanyl]-phenyl}-propionic acid methyl ester

### Example 29

### 4-Fluoro-5-methoxy-3-(3-trifluoromethyl-phenylsulfanyl)-benzo[b]thiophene-2-carboxylic acid (1H-tetrazol-5-yl)-amide

### Example 30

### 3-[2-(Acetyl-methyl-amino)-1-phenyl-propylsulfanyl]-4-fluoro-5-methoxy-benzo[b]thiophene-2-carboxylic acid (1H-tetrazol-5-yl)-amide

### Example 31

### 3-(3-Chloro-phenylsulfanyl)-4-fluoro-5-methoxy-benzo[b]thiophene-2-carboxylic acid (1H-tetrazol-5-yl)-amide

### Example 32

### 4-Fluoro-5-methoxy-3-(3-methoxy-phenylsulfanyl)-benzo[b]thiophene-2-carboxylic acid (1H-tetrazol-5-yl)-amide

### Example 33

### 4-Fluoro-5-methoxy-3-(4-methoxy-phenylsulfanyl)-benzo[b]thiophene-2-carboxylic acid (1H-tetrazol-5-yl)-amide

### Example 34

### 4-[4-Fluoro-5-methoxy-2-(1H-tetrazol-5-ylcarbamoyl)-benzo[b]thiophen-3-ylsulfanyl]-benzoic acid

### Example 35

### {4-[4-Fluoro-5-methoxy-2-(1H-tetrazol-5-ylcarbamoyl)-benzo[b]thiophen-3-ylsulfanyl]-phenyl}-acetic acid

### Example 36

### 3-{4-[4-Fluoro-5-methoxy-2-(1H-tetrazol-5-ylcarbamoyl)-benzo[b]thiophen-3-ylsulfanyl]-phenyl}-propionic acid

### Example 37

### 3-(4-Cyclohexyl-phenylsulfanyl)-4-fluoro-5-methoxy-benzo[b]thiophene-2-carboxylic acid (1H-tetrazol-5-yl)-amide

### Example 38

### 3-Cyclopentylsulfanyl-4-fluoro-5-methoxy-benzo[b]thiophene-2-carboxylic acid (1H-tetrazol-5-yl)-amide

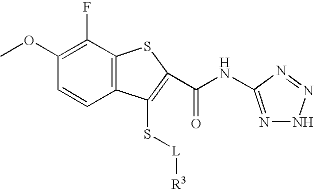

| | | MS | | IC₅₀ |
|---|---|---|---|---|
| Ex. | L—R³ | (M − 1) | NMR^{a} | (μM) |
| 39 | 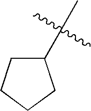 | 392 | δ ppm 1.5 (m, 4 H) 1.6 (m, 2 H) 1.8 (m, 2 H) 3.6 (m, 1H) 3.9 (s, 3 H) 7.8 (d, J = 11.5 Hz, 1 H) 8.0 (d, J = 7.8 Hz, 1 H) 12.2 (s, 1 H) 16.2 (s, 1 H) | 0.016 |
| 40 | 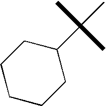 | 406 | δ ppm 1.2 (m, 3 H) 1.3 (m, 2 H) 1.5 (m, 1 H) 1.6 (m, 2 H) 1.8 (m, 2 H) 3.1 (t, J = 10.2 Hz, 1 H) 3.9 (s, 3 H) 7.8 (d, J = 11.5 Hz, 1 H) 7.9 (d, J = 7.8 Hz, 1 H) 12.2 (s, 1 H) 16.3 (s, 1 H) | |

^{a 1}H-NMR (400 MHz, DMSO).

### Example 39

### 3-Cyclopentylsulfanyl-7-fluoro-6-methoxy-benzo[b]thiophene-2-carboxylic acid (1H-tetrazol-5-yl)-amide

In a manner similar to that described in Example 41, Intermediate 20 (0.25 g, 0.67 mmol) cyclopentyl mercaptam (0.214 mL, 2.0 mmol) and 1,8-diazabicyclo{5.4.0)undec-7-ene (DBU) 0.4 mL, 2.68 mmol) in DMF (10 mL) afforded the title compound (0.08 g, 30%). MS: M⁻¹=392.

### Example 40

### 3-Cyclohexylsulfanyl-7-fluoro-6-methoxy -benzo[b]thiophene-2-carboxylic acid (1H-tetrazol-5-yl)-amide

In a manner similar to that described in Example 41, Intermediate 20 (0.2 g, 0.53 mmol) cyclohexyl mercaptam (0.194 mL, 1.59 mmol) and 1,8-diazabicyclo{5.4.0)undec-7-ene (DBU) 0.31 mL, 2.0 mmol) in DMF (10 mL) afforded the title compound (0.14 g, 64%). MS: M⁻¹=406.

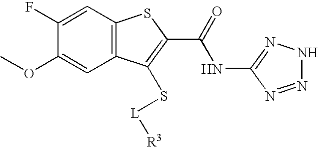

| | | | | IC₅₀ |
|---|---|---|---|---|
| Ex. | L—R³ | MS (M − 1) | NMR^{a} | (μM) |
| 41 | 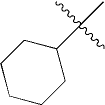 | 406 (M − 1) | δ ppm 1.1 (m, 2 H) 1.2 (m, 1 H) 1.3 (m, 2H) 1.4 (m, 1H) 1.6 (m, 2H) 1.8 (m, 2H) 3.1 (m, 1H) 4.0 (s, 3H) 7.6 (d, J = 8.3 Hz, 1 H) 8.1 (d, J = 10.7 Hz, 1 H) 12.3 (s, 1 H) 16.3 (s, 1 H). | 0.012 |
| 42 | 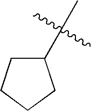 | 392 | δ ppm 1.5 (m, 4H) 1.7 (m, 2H) 1.8 (m, 2 H) 3.6 (m, 1 H) 4.0 (s, 3) 7.6 (d, J = 8.1 Hz, 1H) 8.1 (d, J = 11.0 Hz, 1 H) 12.3 (s, 1 H) 16.3 (s, 1 H). | 0.011 |

^{a 1}H-NMR (400 MHz, DMSO)

### Example 41

### 3-Cyclohexylsulfanyl-6-fluoro-5-methyl-benzo[b]thiophene-2-carboxylic acid (1H-tetrazol-5-yl)-amide

A solution of Intermediate 15 (0.2 g, 0.53 mmol) cyclohexyl mercaptam (0.194 mL, 1.59 mmol) and 1,8-diazabicyclo{5.4.0)undec-7-ene (DBU) 0.63 mL, 2.0 mmol) in DMF (10 mL) was heated to 70° C. for 18 hours. The solvent was removed under reduced pressure and the residue dissolved in DMF/ethanol and treated with 1N HCl until acidic. The title compound was recovered by filtration 0.148 g, 68%). MS: M⁻¹=407.

### Example 42

### 3-Cyclopentylsulfanyl-6-fluoro-5-methoxy-benzo[b]thiophene-2-carboxylic acid (1H-tetrazol-5-yl)-amide

In a manner similar to that described in Example 41, Intermediate 15 (0.25 g, 0.67 mmol) cyclopentyl mercaptam (0.214 mL, 2.0 mmol) and 1,8-diazabicyclo{5.4.0)undec-7-ene (DBU) 0.4 mL, 2.68 mmol) in DMF (10 mL) afforded the title compound (0.23 g, 87%). MS: M⁻¹=392.

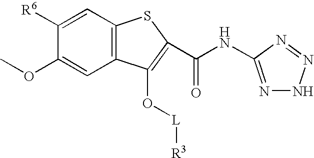

| | | | MS | | IC₅₀ |
|---|---|---|---|---|---|
| Ex. | —L—R³ | R⁶ | (M + 1) | NMR^{a} | (μM) |
| 43 | 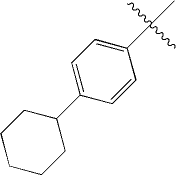 | Br— | 528.0 | δ 1.29 (m, 5H), 1.68 (m, 5H), 2.41 (m, 1H), 3.63 (s, 3H), 6.84 (s, 1H), 6.97 (d, J = 8.55 Hz, 2H), 7.16 (d, J = 8.55 Hz, 2H), 8.45 (s, 1H), 11.72 (s, 1H), 16.07 (s, 1H). | 0.380 |
| 44 | 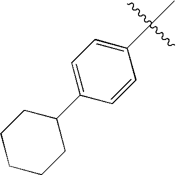 | Cl— | 484.1 | δ 1.30 (m, 5H), 1.70 (m, 5H), 2.42 (m, 1H), 3.66 (s, 3H), 6.89 (s, 1H), 6.99 (d, J = 8.55 Hz, 2H), 7.17 (d, J = 8.55 Hz, 2H), 8.30 (s, 1H), 11.64 (s, 1H), 16.03 (s, 1H). | 0.110 |

^{a 1}H-NMR (400 MHz, DMSO)

### Intermediate 21

### 3-chloro-5-methoxy-6-nitro-benzo[b]thiophene-2-carbonyl chloride

To a 50° C. solution of 3-(3-methoxy-4-nitro-phenyl)-acrylic acid (10.0 g, 44.8 mmol, Chakravarti et al. (1938) J. Chem. Soc. 171-172) and DMAP (4-(dimethylamino)pyridine) (5.5 g) in heptane (50 mL) was added thionyl chloride (20 mL) dropwise via an addition funnel. The reaction was stirred at 100° C. for 18 hours and then EtOAc (400 mL) was added. The reaction was stirred at 100° C. for 18 more hours and then was hot filtered through filter paper. The liquors were concentrated, slurried in hot toluene, and filtered again. The liquors were concentrated to give the title product (11.3 g, 83% yield). MS for sample quenched into K₂CO₃/MeOH: M+1=301.1 (APCI).

### Intermediate 22

### 3-chloro-5-methoxy-6-nitro-benzo[b]thiophene-2-carboxylic acid isopropyl ester

Intermediate 21 (11.2 g, 36.7 mmol) was stirred with DMAP (0.200 g) and triethyl amine (10 mL) in isopropanol (91 mL) at reflux for 16 hours. The reaction mix was concentrated and purified by silica gel flash chromatography [5% EtOAc-hexanes to 50% EtOAc-hexanes gradient elution] to give the title product (6.3 g, 52% yield). MS:M+1=329.1 (APCI).

### Intermediate 23

### 3-chloro-5-methoxy-6-nitro-1-oxo-benzo[b]thiophene-2-carboxylic acid isopropyl ester

To a 0° C. mixture of Intermediate 22 (12.0 g, 36.7 mmol) in TFA (trifluoroacetic acid) (36 mL) and CH₂Cl₂ (36 mL) was added H₂O₂ (30% aq., 6.18 mL, 54.6 mmol) dropwise via syringe. The reaction was stirred at 0° C. for ten minutes and then at room temperature for 90 minutes. The mix was cooled back to 0° C. and was slowly poured into a 0° C. 10% aq. sodium bisulfite solution. The mix was extracted with EtOAc three times. The organic extracts were washed with brine, dried over Na₂SO₄, filtered through celite, and concentrated to give the title product in quantitative yield. MS: M+1=346.0 (APCI).

### Intermediate 24

### 3-(4-cyclohexyl-phenoxy)-5-methoxy-6-nitro-benzo[b]thiophene-2-carboxylic acid isopropyl ester

To a stirring solution of 4-cyclohexyl-phenol (4.42 g, 25.1 mmol) in anhydrous THF (50 mL) was added NaH (0.663 g, 27.6 mmol). The reaction was stirred at room temperature for 3-5 minutes and then was added dropwise to a stirring solution of Intermediate 23 (8.26 g, 23.9 mmol) in anhydrous THF (50 mL). The reaction was stirred at room temperature for 15 minutes and then chlorotrimethylsilane (8.46 mL, 71.7 mmol) and NaI (7.87 g, 52.5 mmol) were added. The reaction was stirred at room temperature for 15 minutes and then was quenched with saturated aq. sodium thiosulfate. The quenched mix was diluted with water and extracted three times with EtOAc. The organics were washed with brine, dried over Na₂SO₄, filtered through celite, and concentrated. The product was purified by silica gel flash chromatography [2.5% Ether-hexanes] to give the title product (8.38 g, 72% yield). MS: M+1=470.1 (APCI).

### Intermediate 25

### 6-amino-3-(4-cyclohexyl-phenoxy)-5-methoxy-benzo[b]thiophene-2-carboxylic acid isopropyl ester

Intermediate 23 (8.37 g, 17.8 mmol) was reduced with Rainey Nickel under H₂ in EtOH in a Parr reactor vessel. The mix was then filtered, concentrated, and purified by silica gel flash chromatography [10% EtOAc-hexanes] to give the title product (5.73 g, 73% yield). MS:M+1=440.2 (APCI).

### Intermediate 26

### 6-bromo-3-(4-cyclohexyl-phenoxy)-5-methoxy-benzo[b]thiophene-2-carboxylic acid isopropyl ester

To a −10° C. solution of Intermediate 25 (0.210 g, 0.478 mmol) in anhydrous CH₂Cl₂ (˜1.5 mL) was added first BF₃.Et₂O ((0.176 mL, 1.43 mmol) and then a solution of t-butyl nitrite (0.068 mL, 0.574 mmol) in CH₂Cl₂ (˜0.5 mL). The reaction was stirred at −10° C. for ten minutes and then was diluted with hexanes until a brown tar oiled out. The liquors were decanted. The residue was dissolved in a minimum of anhydrous CH₃CN and transferred to a stirring 0° C. solution of tetrabutylammonium bromide (Bu₄NBr) (0.110 g, 0.342 mmol) in CH₃CN (2 mL). The reaction was stirred at 0° C. for twenty minutes and then copper (0.015 mg) was added. The reaction was stirred at room temperature for twenty minutes and then the mix was diluted with H₂O and was extracted several times with EtOAc. The organics were washed with brine, dried over Na₂SO₄, filtered through celite, and concentrated. The product was purified by silica gel flash chromatography [10% EtOAc-hexanes] to give the title product (0.114 g, 48% yield). MS: M+1=503.1.

### Intermediate 27

### 6-bromo-3-(4-cyclohexyl-phenoxy)-5-methoxy-benzo[b]thiophene-2-carboxylic acid

Intermediate 26 was saponified as in the synthesis of Intermediate 7 to give the title product.

### Example 43

### 6-Bromo-3-(4-Cyclohexyl-phenoxy)-5-methoxy-benzo[b]thiophene-2-carboxylic acid (2H-tetrazol-5-yl)-amide

Intermediate 27 was coupled with 5-aminotetrazole in a manner similar to that described for the synthesis of Example 7 to give the title product.

### Example 44

### 6-Chloro-3-(4-Cyclohexyl-phenoxy)-5-methoxy-benzo[b]thiophene-2-carboxylic acid (2H-tetrazol-5-yl)-amide

The title product was synthesized in an analogous manner to Example 43 by substituting tetrabutylammonium chloride for tetrabutylammonium bromide.

### Biological Example 1

### PI3Kγ Protein Expression and Purification Protocol

Spodtera frugiperda cells, grown in ESF921 media, were coinfected with baculovirus expressing a glu-tagged p101 and baculovirus expressing an HA-tagged p101γ, at a 3:1 ratio of p101 baculovirus to p110γ baculovirus. Sf9 cells were grown to 1×10⁷ total cells/mL in 10 L bioreactors and harvested 48-72 hours post infection. Samples of infected cells were then tested for expression of p101/p110γ PI3 kinase by immunoprecipitation and Western Blot analysis methods (see below).

To purify PI3Kγ, 4 volumes of room temperature hypotonic lysis buffer (1 mM MgCl₂, 1 mM DTT, 5 mM EGTA, 1 mM Pefabloc, 0.5 μM aprotinin, 5 μM leupeptin, 2 μM pepstatin, 5 μM E64, pH 8) per gram of cell paste, was poured onto frozen cell pellets with stirring, then lysed in a nitrogen “bomb” at 400 psi (599HC T316, Parr Instrument Co, Moline, Ill.). NaCl was added to 150 mM, and sodium cholate was added to 1% and mixed for another 45 minutes. The lysates were clarified by centrifugation for 25 minutes at 14,000 rpm. The lysates were then loaded over anti-glu-linked Protein-G Sepaharose beads (Covance Research Products, Richmond, Calif.) using 20 mL resin/50 g cell paste. The column was washed with 15 volumes of wash buffer (1 mM DTT, 0.2 mM EGTA, 1 mM Pefabloc, 0.5 μM aprotinin, 5 μM leupeptin, 2 μM pepstatin, 5 μM E64, 150 mM NaCl, 1% sodium cholate, pH 8). PI3Kγ was eluted with 6 column volumes of wash buffer that contain 100 μg/mL of a peptide that competes for binding of the glu tag. The column fractions with the eluted protein (determined by taking OD₂₈₀ readings) were collected and dialyzed in 0.2 mM EGTA, 1 mM DTT, 1 mM Pefabloc, 5 μM leupeptin, 0.5% sodium cholate, 150 mM NaCl, and 50% glycerol, pH 8. The fractions were stored at −80° C. until further use.

### Biological Example 2

### G Protein Subunits Expression

Spodtera frugiperda cells were coinfected with baculovirus expressing a glu-tagged G protein β₁ and baculovirus expressing a G protein β₂, at a 1:1 ratio of glu-tagged G protein β₁ baculovirus to G protein β₂ baculovirus. Sf9 cells are grown in 10 L bioreactors and harvested 48-72 hours post infection. Samples of infected cells were tested for G protein β₁/β₂ expression by Western Blot analysis, as described below. Cell lysates were homogenized and loaded onto a column of glu-tagged beads as in Biological Example 1 and competed off the column with a glu peptide and processed as described in Biological Example 1.

### Biological Example 3

### Western Blot Analysis

Protein samples were run on an 8% Tris-Glycine gel and transferred to a 45 μM nitrocellulose membrane. The blots were then blocked with 5% bovine serum albumin (BSA) and 5% ovalburnin in TBST (50 mM Tris, 200 mM NaCl, 0.1% Tween 20, ph 7.4) for 1 hour at room temperature, and incubated overnight at 4° C. with primary antibody diluted 1:1000 in TBST with 0.5% BSA. The primary antibodies for the p110γ, p110α, p110β, p85α, G protein β₁, and G protein γ₂ subunits were purchased from Santa Cruz Biotechnology, Inc., Santa Cruz, Calif. The p101 subunit antibodies were developed at Research Genetics, Inc., Huntsville, Ala. based on a p101 peptide antigen.

After incubation with the primary antibody, the blots were washed in TBST and incubated for 2 hours at room temperaure with goat-anti-rabbit HRP conjugate (Bio-Rad Laboratories, Inc., Hercules, Calif., product Number 170-6515), diluted 1:10,000 in TBST with 0.5% BSA. The antibodies were detected with ECL™ detection reagents (Amersham Biosciences Corp., Piscataway, N.J.) and quantified on a Kodak ISO400F scanner.

### Biological Example 4

### Immunoprecipitation

100 μL of cell paste from Biological Example 1 or 2 was thawed and lysed on ice with 400 μL of hypotonic lysis buffer (25 mM tris, 1 mM DTT, 1 mM EDTA, 1 mM Pefabloc, 5 μM leupeptin, 5 μM E-64 (Roche), 1% Nonidet P40, pH 7.5-8). The lysate was incubated for 2 hours at room temperature with glu-tagged beads (Covance Research Products, Cambridge, England, product Number AFC-115P). The beads were washed 3 times in wash buffer (20 mM Tris, pH 7.8-8, 150 mM NaCl₂, 0.5% NP40) and the protein eluted off the beads by heating in 2 times sample buffer (Invitrogen Corporation, Carlsbad, Calif., product Number LC1676).

### Biological Example 5

### PI3Kγ In Vitro Kinase Assay

The inhibitory properties of the compounds in Table 1 were assayed in an in vitro PI3K assay. In a 96-well polypropylene plate, each well was spotted with 2 μL of 50 times the desired final concentration of compound in DMSO. Purified recombinant p101/p110γ protein (0.03 μg; ˜2.7 nM) and G protein β₁/γ₂ subunits (0.09 μg; ˜57.7 nM) for each reaction was combined in the assay buffer (30 mM HEPES, 100 mM NaCl, 1 mM EGTA, and 1 mM DTT). ATP and [γ-³²P-ATP] (0.09 μCi) were added to this mixture so that the final ATP concentration in the reaction was 20 μM. Lipid micelles were formed by sonicating phosphatidylinositol-4,5-diphosphate (PIP₂), phosphatidylethanolamine (PE), and Na-cholate in the assay buffer for 10 minutes, adding MgCl₂ and incubating on ice for 20 minutes, for final concentrations of 25 μM PIP₂, 300 μM PE, 0.02% Na-cholate, and 10 mM MgCl₂ in the reaction. The reactions were started by adding equal volumes lipid and enzyme mixture in a total volume of 50 μL, allowed to run for 20 minutes at room temperature, and stopped with 100 μL 75 mM H₃PO₄. The lipid product was transferred to a glass fiber filter plate and washed with 75 mM H₃PO₄ several times. The presence of radioactive lipid product (PIP₃) was measured by adding Wallac Optiphase mix to each well and counting in a Wallac 1450 Trilux plate reader (PerkinElmer Life Sciences Inc., Boston, Mass. 02118). The IC₅₀ for each compound tested is reported in μM in the Tables above.

It is understood that the examples and embodiments described herein are for illustrative purposes only and that various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

1. A compound of Formula I: 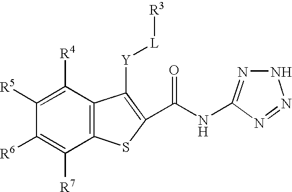
or a pharmaceutically acceptable salt thereof;
wherein Y is O or S;
wherein two of R⁴, R⁵, R⁶, and R⁷ are hydrogen:
wherein one of R⁴, R⁵, R⁶, and R⁷ is selected from the group consisting of: methoxy, C₁-C₃-alkyl-O, CH₂FO, CHF₂O, CF₃O, CF₃CH₂O, or cyclopropyloxy;
wherein one of R⁴, R⁵, R⁶, and R⁷ is F, I, Br, or Cl;
wherein L is absent, a C₁-C₄ alkylene, or 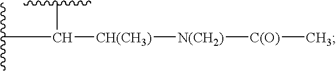
wherein R³ is:
(a) selected from the group consisting of: a C₃-C₈ cycloalkyl, a 5 or 6-membered heterocycloalkyl, a tetrahydropyranyl, and a piperidinyl;
wherein said C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃; or
(b) a phenyl group;
wherein said phenyl group may be optionally substituted with: 1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and —(O)ₘ—C₃-C₈ cycloalkyl;
wherein n is 0, 1 or 2; and
wherein m is 0 or 1.

2. The compound of claim 1, wherein R⁴ and R⁷ are H; R⁵ is methoxy; R⁶ is F; and R³ is a phenyl group;
wherein said phenyl group may be optionally substituted with 1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and —(O)ₘ—C₃-C₈ cycloalkyl;
wherein n is 0, 1 or 2; and
wherein m is 0 or 1.

3. The compound of claim 2, wherein said compound is selected from the group consisting of:
6-Fluoro-3-(4-isopropyl-phenoxy)-5-methoxy-benzo[b]thiophene-2-carboxylic acid (2H-tetrazol-5-yl)-amide;
6-Fluoro-5-methoxy-3-phenoxy-benzo[b]thiophene-2-carboxylic acid iminomethyl-amide;
3-(4-Cyclohexyl-phenoxy)-6-fluoro-5-methoxy-benzo[b]thiophene-2-carboxylic acid (2H-tetrazol-5-yl)-amide;
3-(3-Ethyl-phenoxy)-6-fluoro-5-methoxy-benzo[b]thiophene-2-carboxylic acid (2H-tetrazol-5-yl)-amide;
3-(4-Chloro-phenoxy)-6-fluoro-5-methoxy-benzo[b]thiophene-2-carboxylic acid (2H-tetrazol-5-yl)-amide;
3-(4-Cyclopentyloxy-phenoxy)-6-fluoro-5-methoxy-benzo[b]thiophene-2-carboxylic acid (2H-tetrazol-5-yl)-amide; and
6-Fluoro-5-methoxy-3-phenylsulfanyl-benzo[b]thiophene-2-carboxylic acid (1H-tetrazol-5-yl)-amide.

4. The compound of claim 1, wherein R⁴ and R⁷ are H; R⁵ is methoxy; R⁶ is F; and R³ is an R³ is selected from the group consisting of a C₃-C₈ cycloalkyl, a 5 or 6-membered heterocycloalkyl, a tetrahydropyranyl, and a piperidinyl;
wherein said C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃.

5. The compound of claim 4, wherein said compound is selected from the group consisting of:
3-(3,5-Dimethyl-cyclohexyloxy)-6-fluoro-5-methoxy-benzo[b]thiophene-2-carboxylic acid (2H-tetrazol-5-yl)-amide;
3-Cycloheptyloxy-6-fluoro-5-methoxy-benzo[b]thiophene-2-carboxylic acid (2H-tetrazol-5-yl)-amide;
cis-(±)-6-Fluoro-5-methoxy-3-(3-methyl-cyclohexyloxy)-benzo[b]thiophene-2-carboxylic acid (2H-tetrazol-5-yl)-amide;
6-Fluoro-5-methoxy-3-(3,3,5-trimethyl-cyclohexyloxy)-benzo[b]thiophene-2-carboxylic acid (2H-tetrazol-5-yl)-amide;
3-(3,3-Dimethyl-cyclohexyloxy)-6-fluoro-5-methoxy-benzo[b]thiophene-2-carboxylic acid (2H-tetrazol-5-yl)-amide;
3-Cyclohexyloxy-6-fluoro-5-methoxy-benzo[b]thiophene-2-carboxylic acid (2H-tetrazol-5-yl)-amide;
6-Fluoro-5-methoxy-3-(1-methyl-cyclopropylmethoxy)-benzo[b]thiophene-2-carboxylic acid (2H-tetrazol-5-yl)-amide;
4-[6-Fluoro-5-methoxy-2-(2H-tetrazol-5-ylcarbamoyl)-benzo[b]thiophen-3-yloxy]-piperidine-1-carboxylic acid tert-butyl ester;
3-Cyclopentylsulfanyl-6-fluoro-5-methoxy-benzo[b]thiophene-2-carboxylic acid (1H-tetrazol-5-yl)-amide; and
3-Cyclohexylsulfanyl-6-fluoro-5-methyl-benzo[b]thiophene-2-carboxylic acid (1H-tetrazol-5-yl)-amide.

6. The compound of claim 1, wherein R⁶ and R⁷ are H; R⁵ is methoxy; R⁴ is F; and R³ is selected from the group consisting of a C₃-C₈ cycloalkyl, a 5 or 6-membered heterocycloalkyl, a tetrahydropyranyl, and a piperidinyl;
wherein said C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃.

7. The compound of claim 1, wherein R⁴ and R⁵ are H; R⁶is methoxy; R⁷ is F; and R³ is a phenyl group;
wherein said phenyl group may be optionally substituted with: 1 to 3 substituents independently selected from the group consisting of:
Br, F, Cl, —CF₃, —OH, C₁-C₄ alkyl, —O—C₁-C₆alkyl, —(CH₂)ₙ—C(O)—O—CH₃, (CH₂)ₙ—C(O)—OH, and —(O)ₘ—C₃-C₈ cycloalkyl;
wherein n is 0, 1 or 2; and
wherein m is 0 or 1.

8. The compound of claim 1, wherein R⁴ and R⁵ are H; R⁶is methoxy; R⁷ is F; Y is S; and R³ is selected from the group consisting of a C₃-C₈ cycloalkyl, a 5 or 6-membered heterocycloalkyl, a tetrahydropyranyl, and a piperidinyl;
wherein said C₃-C₈ cycloalkyl, 5 or 6-membered heterocycloalkyl, tetrahydropyranyl, and a piperidinyl may be optionally substituted with 1, 2, 3, or 4 methyls, or —C(O)—O—C(CH₃)₃.

9. The compound of claim 8, wherein said compound is selected from the group consisting of:
3-Cyclopentylsulfanyl-7-fluoro-6-methoxy-benzo[b]thiophene-2-carboxylic acid (1H-tetrazol-5-yl)-amide; and
3-Cyclohexylsulfanyl-7-fluoro-6-methoxy-benzo[b]thiophene-2-carboxylic acid (1H-tetrazol-5-yl)-amide.

10. A method of treating a subject comprising:
administering, to a subject suffering from a disease selected from the group consisting of: rheumatoid arthritis, osteoarthritis, psoriatic arthritis, psoriasis, ankylosing spondylitis, inflammatory diseases, and autoimmune diseases, a pharmaceutical composition comprising a therapeutically effective amount of a compound of claim 1 and a pharmaceutically acceptable carrier.

11. The method of claim 10, wherein said disease is rheumatoid arthritis.

12. The method of claim 11, wherein said compound is a compound of any one of claims 1-9.

13. A method of treating a subject comprising:
administering, to a subject suffering from a disease selected from the group consisting of: cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, coronary artery disease, cancer, breast cancer, gliobastoma, endometrial carcinoma, hepatocellular carcinoma, colon cancer, lung cancer, melanoma, renal cell carcinoma, thyroid carcinoma, small cell lung cancer, squamous cell lung carcinoma, glioma, prostate cancer, ovarian cancer, cervical cancer, leukemia, cell lymphoma, lymphoproliferative disorders, respiratory diseases, bronchitis, asthma, and chronic obstructive pulmonary disease, a pharmaceutical composition comprising a therapeutically effective amount of a compound of claim 1 and a pharmaceutically acceptable carrier. group.

14. A pharmaceutical composition comprising:
a therapeutically effective amount of a compund of claim 1 and a pharmaceutically acceptable carrier.

15. A pharmaceutical composition comprising:
a pharmaceutically acceptable carrier; and
a therapeutically effective amount of a compund or a pharmaceutically acceptable salt thereof selected from the group consisting of:
6-Fluoro-3-(4-isopropyl-phenoxy)-5-methoxy-benzo[b]thiophene-2-carboxylic acid (2H-tetrazol-5-yl)-amide;
6-Fluoro-5-methoxy-3-phenoxy-benzo[b]thiophene-2-carboxylic acid iminomethyl-amide;
3-(4-Cyclohexyl-phenoxy)-6-fluoro-5-methoxy-benzo[b]thiophene-2-carboxylic acid (2H-tetrazol-5-yl)-amide;
3-(3-Ethyl-phenoxy)-6-fluoro-5-methoxy-benzo[b]thiophene-2-carboxylic acid (2H-tetrazol-5-yl)-amide;
3-(4-Chloro-phenoxy)-6-fluoro-5-methoxy-benzo[b]thiophene-2-carboxylic acid (2H-tetrazol-5-yl)-amide;
3-(4-Cyclopentyloxy-phenoxy)-6-fluoro-5-methoxy-benzo[b]thiophene-2-carboxylic acid (2H-tetrazol-5-yl)-amide;
3-(3,5-Dimethyl-cyclohexyloxy)-6-fluoro-5-methoxy-benzo[b]thiophene-2-carboxylic acid (2H-tetrazol-5-yl)-amide;
3-Cycloheptyloxy-6-fluoro-5-methoxy-benzo[b]thiophene-2-carboxylic acid (2H-tetrazol-5-yl)-amide;
cis-(±)-6-Fluoro-5-methoxy-3-(3-methyl-cyclohexyloxy)-benzo[b]thiophene-2-carboxylic acid (2H-tetrazol-5-yl)-amide;
6-Fluoro-5-methoxy-3-(3,3,5-trimethyl-cyclohexyloxy)-benzo[b]thiophene-2-carboxylic acid (2H-tetrazol-5-yl)-amide;
3-(3,3-Dimethyl-cyclohexyloxy)-6-fluoro-5-methoxy-benzo[b]thiophene-2-carboxylic acid (2H-tetrazol-5-yl)-amide;
3-Cyclohexyloxy-6-fluoro-5-methoxy-benzo[b]thiophene-2-carboxylic acid (2H-tetrazol-5-yl)-amide;
6-Fluoro-5-methoxy-3-(1-methyl-cyclopropylmethoxy)-benzo[b]thiophene-2-carboxylic acid (2H-tetrazol-5-yl)-amide;
4-[6-Fluoro-5-methoxy-2-(2H-tetrazol-5-ylcarbamoyl)-benzo[b]thiophen-3-yloxy]-piperidine-1-carboxylic acid tert-butyl ester;
6-Fluoro-5-methoxy-3-phenylsulfanyl-benzo[b]thiophene-2-carboxylic acid (1H-tetrazol-5-yl)-amide;
3-Cyclopentylsulfanyl-6-fluoro-5-methoxy-benzo[b]thiophene-2-carboxylic acid (1H-tetrazol-5-yl)-amide;
3-Cyclohexylsulfanyl-6-fluoro-5-methyl-benzo[b]thiophene-2-carboxylic acid (1H-tetrazol-5-yl)-amide;
3-Cyclopentylsulfanyl-7-fluoro-6-methoxy-benzo[b]thiophene-2-carboxylic acid (1H-tetrazol-5-yl)-amide; and
3-Cyclohexylsulfanyl-7-fluoro-6-methoxy-benzo[b]thiophene-2-carboxylic acid (1H-tetrazol-5-yl)-amide.
